(19) United States
(12) Patent Application Publication
(10) Pub. No.: US 2015/0343100 A1
(43) Pub. Date: Dec. 3, 2015

(54) BIMODAL FLUOROPHORE-LABELED LIPOSOMES AND ASSOCIATED METHODS AND SYSTEMS

(71) Applicant: Memorial Sloan Kettering Cancer Center, New York, NY (US)

(72) Inventors: Carlos Perez-Medina, New York, NY (US); Thomas Reiner, New York, NY (US); Jason S. Lewis, New York, NY (US); Willem J.M. Mulder, New York, NY (US); Zahi A. Fayad, New York, NY (US)

(21) Appl. No.: 14/722,425

(22) Filed: May 27, 2015

Related U.S. Application Data
(60) Provisional application No. 62/004,174, filed on May 28, 2014.
(60) Provisional application No. 62/008,999, filed on Jun. 6, 2014.

(51) Int. Cl. A61K 51/12; A61K 49/00; A61K 51/04; A61K 31/704

(52) U.S. Cl. CPC A61K 51/1234; A61K 31/704; A61K 49/0032; A61K 51/0478

(57) ABSTRACT

Described herein is a non-invasive quantitative positron emission tomography (PET) nanoreporter technology that allows personalized therapeutic outcome prediction. In a breast cancer mouse model, it was demonstrated that co-injecting Doxil and a Zirconium-89 nanoreporter (⁸⁹Zr-NRep) enabled highly precise doxorubicin (DOX) quantification. Imaging ⁸⁹Zr-NRep via PET revealed remarkable Doxil accumulation heterogeneity independent of tumor size.

### CROSS-REFERENCE TO RELATED APPLICATIONS

This application claims priority to and the benefit of, and incorporates herein by reference in their entireties, U.S. Provisional Patent Application Ser. Nos. 62/004,174 and 62/008,999, filed May 28, 2014 and Jun. 6, 2014, respectively.

### GOVERNMENT SUPPORT

This work was supported by National Institutes of Health grants NIH 1 R01 HL125703 (W. J. M. M.) R01 CA155432 (W. J. M. M.) K25 EB016673 (T. R.) AND P30 CA008748.

### FIELD OF INVENTION

This invention relates generally to a liposome labeled with a fluorophore and a radioisotope, and related imaging systems and methods. In particular embodiments, the invention relates to a non-invasive quantitative positron emission tomography (PET) nanoreporter technology that provides personalized therapeutic outcome prediction.

### BACKGROUND

Clinically approved nanoparticle drug formulations such as Doxil® or Abraxane® are used to treat a wide range of cancers (e.g., ovarian cancer, breast cancer and lung cancer) and are increasingly becoming integrated in clinical cancer care. Moreover, numerous benefits of nanotherapies include enhanced pharmacokinetics, increased drug stability, and improved tumor bioavailability.

Despite the remarkable potential growth of nanomedicine over the last three decades, its clinical benefits vary patient-to-patient. Therefore, identifying patients amenable to anti-cancer nanotherapy should be based on individualized inclusion criteria derived from quantifiable procedures. To this end, non-invasive imaging (e.g., imaging of (super)paramagnetic labels using magnetic resonance imaging, or e.g., imaging of radioisotopes using nuclear imaging) can aid in patient-specific nanomedicine by allowing swift adjustments in dosage and/or treatment regimen. However, traditional clinical protocols for non-invasive imaging lack specificity and many experimental imaging-facilitated nanotherapy assessment studies have little translational potential. Moreover, tumor heterogeneity and variable vascular permeability between patients overcomes any potential benefit that non-invasive imaging may offer. Such approaches potentially compromise the functionality of the nanotherapy and their translation and clinical implementation are far too expensive for general use.

Thus, there is a need for compositions and systems for screening nanoparticle uptake on a patient-to-patient basis. In particular, there is a need for compositions and systems that are easy-to-prepare and compatible with clinically approved anti-cancer nanotherapy (e.g., the compositions and systems must exhibit the same pharmacokinetic signature as the clinically approved nanotherapies).

### SUMMARY OF INVENTION

Described herein is a non-invasive quantitative positron emission tomography (PET) nanoreporter technology that allows personalized therapeutic outcome prediction. Moreover, described herein are dual-labeled liposomes, and methods and systems for their use as diagnostic tools, e.g., to screen individual subjects for nanotherapy amenability and biodistribution. For example, a stable liposome platform can be efficiently labeled with the radioisotope ⁸⁹Zr and a fluorophore, such as a Cy5 analog or a Cy7 analog. These liposomes accumulate in vascularized tumor areas via the EPR effect and can be used as companion imaging agents to stratify patients into their appropriate treatment groups. For example, use of ⁸⁹Zr-NRep PET imaging revealed remarkable Doxil accumulation heterogeneity independent of tumor size.

The labeled liposomes described herein are useful in the research, diagnosis, and treatment of diseases, particularly those implicating the enhanced permeability and retention (EPR) effect and/or other passive targeting mechanisms. These include cancer, cardiovascular disease, and infection/inflammation disorders. The liposomes can also be used intraoperatively to delineate malignancy in cancer in a variety of tumors, since it is not an actively targeted probe. A PET scan prior to surgery can clarify the utility of the probe at the operating table, since it would give reliable information about the extent of accumulation. Optical imaging equipment with wavelength filters tuned to the emission wavelength(s) of the fluorophore would visualize malignant areas, which aids surgical procedures in real time.

The liposomes and associated methods are useful to evaluate the performance of liposomal therapeutics to infer safety and efficacy of treatment. They provide for non-invasive visualization of an injected dose. Physicians are provided with valuable information regarding risks and benefits of a given nanotherapeutic on an individual, personalized basis, and can be used for surgical planning and intraoperative guidance for a wide array of cancers.

In one aspect, the invention is directed to a desferrioxamine-bearing liposome (DFO-L) labeled with ⁸⁹Zr and a fluorophore, wherein the ⁸⁹Zr is attached to a surface of the liposome via a chelating moiety. In certain embodiments, the chelating moiety is lipid-based and/or comprises a lipophilic anchor group. In certain embodiments, the chelating moiety comprises a phospholipid-chelator. In certain embodiments, the phospholipid-chelator comprises 1,2-distearoyl-sn-glycero-3-phosphoethanolamine (DSPE)-desferrioxamine (DFO). In certain embodiments, the fluorophore comprises a NIR (near infrared) dye. In certain embodiments, the NIR dye is Cy5 or Cy7.

In another aspect, the invention is directed to a dibenzoazacyclooctyne-bearing liposome (DBCO-L) labeled with ⁸⁹Zr and a fluorophore, wherein the ⁸⁹Zr is attached to a surface of the liposome via a clickable moiety. the clickable moiety comprises a bioorthogonal group (e.g., trasn-cyclooctene, tetrazine, alkyne, strained alkene, thiol, DFO-azide, and maleimide). In certain embodiments, the clickable moiety comprises a DFO-azide group. In certain embodiments, the fluorophore comprises a NIR dye. In certain embodiments, the NIR dye is Cy5 and/or Cy7. In certain embodiments, the liposome has a mean diameter from about 10 nm to about 1 μm (e.g., from 25 nm to 500 nm, from 50 nm to about 300 nm, from 75 nm to 150 nm, from 10 nm to 25 nm, or from 500 nm to 1 μm, e.g., about 100 nm).

In another aspect, the invention is directed to a method of treating a disease or disorder, the method comprising: administering a desferrioxamine-bearing liposome (DFO-L) labeled with ⁸⁹Zr and a fluorophore to a subject, wherein the ⁸⁹Zr is attached to a surface of the liposome via a chelating moiety. In certain embodiments, the chelating moiety is lipid-based and/or comprises a lipophilic anchor group. In certain embodiments, the chelating moiety comprises a phospholipid-chelator. In certain embodiments, the phospholipid-chelator is 1,2-distearoyl-sn-glycero-3-phosphoethanolamine (DSPE)-desferrioxamine (DFO). In certain embodiments, the fluorophore comprises a NIR (near infrared) dye. In certain embodiments, the NIR dye is a Cy5 and/or Cy7.

In certain embodiments, the method further comprises: capturing and displaying a positron emission tomography (PET) image of a tissue of the subject comprising the radiolabeled liposome. In certain embodiments, the method further comprises: capturing and displaying an optical image of a tissue of the subject comprising the radiolabeled liposome. In certain embodiments, the method further comprises: capturing and displaying a sequence of PET images in real time. In certain embodiments, the method further comprises capturing and displaying a sequence of optical images in real time. In certain embodiments, the sequence of optical images is a sequence of fluorescence images. In certain embodiments, the capturing and displaying the positron emission tomography (PET) image of a tissue of the subject comprising the radiolabeled liposome and the capturing and displaying the optical image of the tissue of the subject comprising the radiolabeled liposome are performed contemporaneously. In certain embodiments, the capturing and displaying the positron emission tomography (PET) image of a tissue of the subject comprising the radiolabeled liposome and the capturing and displaying the optical image of the tissue of the subject comprising the radiolabeled liposome are conducted during a surgical procedure.

In another aspect, the invention is directed to a method of testing loading and/or delivery potential of a bimodal-labeled liposome in a tissue of a subject, the method comprising: (a) administering the bimodal-labeled liposome, wherein the bimodal-labeled liposome is labeled with a radioisotope and a near infrared (NIR) dye, wherein the NIR dye comprises a lipophilic drug-mimic to test loading and/or delivery potential of the liposome; (b) capturing and displaying a positron emission tomography (PET) image of the tissue of the subject comprising the radiolabeled liposome; and (c) capturing and displaying an optical image of the tissue of the subject comprising the radiolabeled liposome.

In certain embodiments, the bimodal-labeled liposome is a desferrioxamine-bearing liposome (DFO-L) labeled with ⁸⁹Zr and a fluorophore, wherein the ⁸⁹Zr is attached to a surface of the liposome via a chelating moiety. In certain embodiments, the chelating moiety is lipid-based and/or comprises a lipophilic anchor group. In certain embodiments, the chelating moiety comprises a phospholipid-chelator. In certain embodiments, the phospholipid-chelator is 1,2-distearoyl-sn-glycero-3-phosphoethanolamine (DSPE)-desferrioxamine (DFO). In certain embodiments, the method further comprises capturing and displaying a sequence of PET images in real time.

In certain embodiments, the NIR dye comprises Cy5 and/or Cy7. In certain embodiments, the optical image comprises a fluorescence image.

In certain embodiments, the method further comprises capturing and displaying a sequence of optical images in real time. In certain embodiments, the method further comprises capturing and displaying a first PET image is performed at 24 hours after administration.

In certain embodiments, the method further comprises (d) administering a second bimodal-labeled liposome comprising a therapeutic, wherein the bimodal-labeled liposome is labeled with a radioisotope and a fluorophore. In certain embodiments, the bimodal-labeled liposome is a desferrioxamine-bearing liposome (DFO-L) labeled with ⁸⁹Zr and a fluorophore, wherein the ⁸⁹Zr is attached to a surface of the liposome via a chelating moiety. In certain embodiments, the chelating moiety is lipid-based and/or comprises a lipophilic anchor group. In certain embodiments, the chelating moiety comprises a phospholipid-chelator. In certain embodiments, the phospholipid-chelator is 1,2-distearoyl-sn-glycero-3-phosphoethanolamine (DSPE)-desferrioxamine (DFO). In certain embodiments, the therapeutic comprises doxorubicin. In certain embodiments, the second bimodal-labeled liposome is doxorubicin HCl liposome.

In certain embodiments, the fluorophore comprises a NIR dye. In certain embodiments, the NIR dye is Cy5 and/or Cy7.

In certain embodiments, the method further comprises (e) capturing and displaying a positron emission tomography (PET) image of the tissue of the subject comprising the radiolabeled liposome comprising the therapeutic; and/or (f) capturing and displaying an optical image of the tissue of the subject comprising the radiolabeled liposome comprising the therapeutic.

### Definitions

In order for the present disclosure to be more readily understood, certain terms are first defined below. Additional definitions for the following terms and other terms are set forth throughout the specification.

In this application, the use of “or” means “and/or” unless stated otherwise. As used in this application, the term “comprise” and variations of the term, such as “comprising” and “comprises,” are not intended to exclude other additives, components, integers or steps. As used in this application, the terms “about” and “approximately” are used as equivalents. Any numerals used in this application with or without about/approximately are meant to cover any normal fluctuations appreciated by one of ordinary skill in the relevant art. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

“Administration”: The term “administration” refers to introducing a substance into a subject. In general, any route of administration may be utilized including, for example, parenteral (e.g., intravenous), oral, topical, subcutaneous, peritoneal, intraarterial, inhalation, vaginal, rectal, nasal, introduction into the cerebrospinal fluid, or instillation into body compartments. In some embodiments, administration is oral. Additionally or alternatively, in some embodiments, administration is parenteral. In some embodiments, administration is intravenous.

“Biocompatible”: The term “biocompatible”, as used herein is intended to describe materials that do not elicit a substantial detrimental response in vivo. In certain embodiments, the materials are “biocompatible” if they are not toxic to cells. In certain embodiments, materials are “biocompatible” if their addition to cells in vitro results in less than or equal to 20% cell death, and/or their administration in vivo does not induce inflammation or other such adverse effects. In certain embodiments, materials are biodegradable.

“Biodegradable”: As used herein, “biodegradable” materials are those that, when introduced into cells, are broken down by cellular machinery (e.g., enzymatic degradation) or by hydrolysis into components that cells can either reuse or dispose of without significant toxic effects on the cells. In certain embodiments, components generated by breakdown of a biodegradable material do not induce inflammation and/or other adverse effects in vivo. In some embodiments, biodegradable materials are enzymatically broken down. Alternatively or additionally, in some embodiments, biodegradable materials are broken down by hydrolysis. In some embodiments, biodegradable polymeric materials break down into their component polymers. In some embodiments, breakdown of biodegradable materials (including, for example, biodegradable polymeric materials) includes hydrolysis of ester bonds. In some embodiments, breakdown of materials (including, for example, biodegradable polymeric materials) includes cleavage of urethane linkages.

“Carrier”: As used herein, “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Suitable pharmaceutical carriers are described in “Remington's Pharmaceutical Sciences” by E. W. Martin.

In some embodiments, click reactive groups are used (for ‘click chemistry’). Examples of click reactive groups include the following: alkyne, azide, thiol (sulfydryl), alkene, acrylate, oxime, maliemide, NHS (N-hydroxysuccinimide), amine (primary amine, secondary amine, tertiary amine, and/or quarternary ammonium), phenyl, benzyl, hydroxyl, carbonyl, aldehyde, carbonate, carboxylate, carboxyl, ester, methoxy, hydroperoxy, peroxy, ether, hemiacetal, hemiketal, acetal, ketal, orthoester, orthocarbonate ester, amide, carboxyamide, imine (primary ketimine, secondary ketamine, primary aldimine, secondary aldimine), imide, azo (diimide), cyanate (cyanate or isocyanate), nitrate, nitrile, isonitrile, nitrite (nitrosooxy group), nitro, nitroso, pyridyl, sulfide, disulfide, sulfinyl, sulfonyl, sulfino, sulfo, thiocyanate, isothiocyanate, caronothioyl, thione, thial, phosphine, phosphono, phosphate, phosphodiester, borono, boronate, bornino, borinate, halo, fluoro, chloro, bromo, and/or iodo moieties.

“Radiolabel”: As used herein, “radiolabel” refers to a moiety comprising a radioactive isotope of at least one element. Exemplary suitable radiolabels include but are not limited to those described herein. In some embodiments, a radiolabel is one used in positron emission tomography (PET). In some embodiments, a radiolabel is one used in single-photon emission computed tomography (SPECT). In some embodiments, radioisotopes comprise ^{99m}Tc, ¹¹¹In, ⁶⁴Cu, ⁶⁷Ga, ¹⁸⁶Re, ¹⁸⁸Re, ¹⁵³Sm, ¹⁷⁷Lu, ⁶⁷Cu, ¹²³I, ¹²⁴I, ¹²⁵I, ¹¹C, ¹3N, ¹⁵O, ¹⁸F, ¹⁸⁶Re, ¹⁸⁸Re, ¹⁵³Sm, ¹⁶⁶Ho, ¹⁷⁷Lu, ¹⁴⁹Pm, ⁹⁰Y, ²¹³Bi, ¹⁰³Pd, ¹⁰⁹Pd, ¹⁵⁹Gd, ¹⁴⁰La, ¹⁹⁸Au, ¹⁹⁹Au, ¹⁶⁹Yb, ¹⁷⁵Yb, ¹⁶⁵Dy, ¹⁶⁶Dy, ⁶⁷Cu, ¹⁰⁵Rh, ¹¹¹Ag, ⁸⁹Zr, ²²⁵Ac, and ¹⁹²Ir.

“Subject”: As used herein, the term “subject” includes humans and mammals (e.g., mice, rats, pigs, cats, dogs, and horses). In many embodiments, subjects are be mammals, particularly primates, especially humans. In some embodiments, subjects are livestock such as cattle, sheep, goats, cows, swine, and the like; poultry such as chickens, ducks, geese, turkeys, and the like; and domesticated animals particularly pets such as dogs and cats. In some embodiments (e.g., particularly in research contexts) subject mammals will be, for example, rodents (e.g., mice, rats, hamsters), rabbits, primates, or swine such as inbred pigs and the like.

“Therapeutic agent”: As used herein, the phrase “therapeutic agent” refers to any agent that has a therapeutic effect and/or elicits a desired biological and/or pharmacological effect, when administered to a subject.

“Treatment”: As used herein, the term “treatment” (also “treat” or “treating”) refers to any administration of a substance that partially or completely alleviates, ameliorates, relives, inhibits, delays onset of, reduces severity of, and/or reduces incidence of one or more symptoms, features, and/or causes of a particular disease, disorder, and/or condition. Such treatment may be of a subject who does not exhibit signs of the relevant disease, disorder and/or condition and/or of a subject who exhibits only early signs of the disease, disorder, and/or condition. Alternatively or additionally, such treatment may be of a subject who exhibits one or more established signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors that are statistically correlated with increased risk of development of the relevant disease, disorder, and/or condition.

Drawings are presented herein for illustration purposes, not for limitation.

### BRIEF DESCRIPTION OF DRAWINGS

The foregoing and other objects, aspects, features, and advantages of the present disclosure will become more apparent and better understood by referring to the following description taken in conjunction with the accompanying drawings, in which:

FIG. 1A shows a schematic representation of the FDA-approved Doxil nanoformulation (left) and the 89Zr-NRep doped Doxil nanoformulation used in the study (right).

FIG. 1B shows an exemplary embodiment of a liposomal nanoreporter ⁸⁹Zr-NRep modified with ⁸⁹Zr-chelating desferrioxamine (DFO).

FIG. 1C shows a comparison of size exclusion retention times for clinical grade Doxil nanoformulation (fluorescence emission) and ⁸⁹Zr-NRep (HPLC γ-counter).

FIG. 1D shows a schematic indicating that co-injecting ⁸⁹Zr-NRep and Doxil allows for non-invasive quantification of DOX delivery.

FIG. 1E shows a correlation between ⁸⁹Zr-NRep (% ID/g) and DOX (% ID_{eq}./g) uptake. Data points represent tumor tissue from mice sacrificed at 6 h, 24 h, or 48 h post administration. Tissues were excised, and associated activity counted (γ-counter), before DOX was extracted from the tissues.

FIGS. 2A-2C show ⁸⁹Zr-NRep mirrors DOX accumulation in tumor. Curves show correlation between radioactivity and doxorubicin fluorescence determined ex vivo in digested tumor samples at 6 h (FIG. 2A), 24 h (FIG. 2B), and 48 h (FIG. 2C) after co-injection of Doxil and ⁸⁹Zr-NRep.

FIG. 3A shows representative images of 4T1 tumor bearing mice with low ⁸⁹Zr-NRep uptake (mouse A, left) and high ⁸⁹Zr-NRep uptake (mouse B, right).

FIG. 3B shows a correlation of uptake values generated by non-invasive PET imaging and DOX tumor concentrations.

FIG. 3C shows a correlation of tumor-associated activity (measured ex vivo, γ-counter) and DOX tumor concentrations.

FIG. 4A depicts tumor growth for the different groups of 4T1 tumor-bearing female NCr nude mice used in the therapeutic study, showing tumor volume (mm³) vs. time (days post injection).

FIG. 4B depicts tumor growth for the different groups of 4T1 tumor-bearing female NCr nude mice used in the therapeutic study, showing relative tumor increase after administration of the corresponding doses.

FIG. 4C depicts tumor growth for the different groups of 4T1 tumor-bearing female NCr nude mice used in the therapeutic study, showing average cumulative daily tumor growth rates.

FIG. 4D shows survival curve for the different groups in the therapeutic study.

FIG. 5A shows individual non-invasively determined intratumoral DOX concentrations for mice receiving either 20 mg/kg Doxil (N=20) or 10 mg/kg Doxil (N=10).

FIG. 5B shows uptake values obtained for mice receiving 20 mg/kg Doxil (N=20) versus initial tumor volumes. Labeled red arrows in FIGS. 5A and 5B indicate data points for mice HD-10, HD-07 and HD-18.

FIG. 5C shows a PET scans of mice HD-10 (large tumor, high uptake), HD-07 (small tumor, high uptake) and HD-18 (medium-sized tumor, low uptake), demonstrating intertumor uptake heterogeneity.

FIG. 6A shows individual tumor size increase in mouse cohorts treated with 20 mg/kg Doxil and greater than 25 mg/kg intratumoral DOX concentration (N=9); less than 25 mg/kg intratumoral Doxil concentration (N=11) and controls (N=15).

FIG. 6B shows mean values of the groups in FIG. 6A.

FIG. 6C shows a comparison of tumor growth rates for mice treated with 20 mg/kg Doxil that received greater than 25 mg/kg intratumoral DOX (N=9), less than 25 mg/kg DOX (N=11) or 10 mg/kg Doxil (N=10) at 2 days (left), 7 days (middle) and 12 days (right) post-treatment. The data from 2 days represent the initial daily growth rate (day 0-2, left); the 7-and 12-day data are the average daily growth rates from day 2 onwards.

FIG. 6D shows mean values of the average daily growth rates from day 2 onwards.

FIGS. 6E and 6F illustrate a Kaplan-Meier plot and table showing the survival and mean survival of individual mouse cohorts treated with 20 mg/kg Doxil (N=20), 10 mg/kg Doxil (N=10), 20 mg/kg Doxil and greater than 25 mg intratumoral DOX/kg (N=9) or less than 25 mg intratumoral DOX/kg (N=11), plus the PBS treated control group (N=15). Error bars are mean±SEM. P-values were calculated with Student's t-tests, unpaired; ns=not significant, *p<0.05, **p<0.01, ***p<0.001, ****p<0.0001.

FIG. 7 shows that DOX concentration in tumors, as determined by ⁸⁹Zr-NRep PET imaging, inversely correlates with tumor growth rate. Significant inverse correlations between the non-invasively determined amounts of DOX delivered to tumors can be observed at 7, 9, 12 and 14 days post administration.

FIGS. 8A-8C show strategies for the labeling of liposomes with ⁸⁹Zr by click labeling (FIG. 8A) and surface chelation (FIG. 8B).

FIG. 8C shows, in some embodiments, lipid composition of the liposomes as described herein.

FIG. 9 depicts synthesis of the building blocks 3, 6 and 8, and corresponding mass spectra.

FIG. 10A depicts sizes (expressed as mean effective diameter) and polydispersity values of the different liposomes described in the present work.

FIG. 10B depicts size exclusion chromatogram showing absorption at 650 nm (front) and radioactive trace (back) of a sample of DilC@89Zr-SCL.

FIG. 11A shows radiosynthesis of [89Zr]9.

FIG. 11B shows HPLC chromatograms showing UV (absorption at 220 nm, front) and radioactive (back) traces of a mixture of [89Zr]9 and the reference compound 9, demonstrating co-elution.

FIG. 12A shows size exclusion chromatograms showing the radioactive traces of ⁸⁹Zr-CLL (front) and ⁸⁹Zr-9 (back).

FIG. 12B illustrates size exclusion chromatograms showing the radioactive traces of ⁸⁹Zr-SCL (front) and ⁸⁹Zr-oxalate (back).

FIG. 12C shows in vitro serum stability of ⁸⁹Zr-CLL and ⁸⁹Zr-SCL.

FIG. 13A shows pharmacokinetics of ⁸⁹Zr-SCL and ⁸⁹Zr-CLL (n=3).

FIG. 13B shows radioactivity distribution in selected tissues of ⁸⁹Zr-SCL (left) and ⁸⁹Zr-CLL (right) (n greater than or equal to 3).

FIG. 13C shows PET/CT imaging of ⁸⁹Zr-SCL: CT only (left), PET/CT fusion (middle) and 30 rendering PET/CT fusion (right) at 24 h p.i.

FIG. 14A shows PET-quantified radioactivity distribution in selected tissues of 89Zr-SCL (left) and DilC@89Zr-SCL (right), expressed as % ID/g±SD (n greater than or equal to 3).

FIG. 14B shows whole-body near infrared fluorescence imaging (λ_{Ex}=650 nm/λ_{Em}=670 nm) at 24 h after administration of DilC@89Zr-SCL (left) and 89Zr-SCL (right), which was used as control.

FIG. 14C shows near infrared fluorescence imaging (λ_{Ex}=650 nm/λ_{Em}=670 nm) of excised specimens of muscle, tumor, liver and spleen (from left to right) collected at 24 h after administration of 89Zr-SCL (top two dishes) and DilC@89Zr-SCL (bottom two dishes).

FIG. 15 shows PET/CT imaging of ⁸⁹Zr-CLL showing CT only (left) and PET/CT fusion (right) at 24 h p.i. The arrow indicates the location of the tumor.

FIG. 16A shows a schematic of the dual-labeled liposomes DilC@89Zr-SCL.

FIG. 16B shows whole-body NIR fluorescence imaging (λ_{Ex}=650 nm/λ_{Em}=670 nm) (left) and 3D rendering PET/CT fusion image (right) of the same animal at 24 h after administration of DilC@89Zr-SCL.

FIG. 16C shows tumor sections showing autoradiography (top) and confocal microscopy at 670 nm (bottom).

FIG. 16D shows a comparison of near infrared fluorescence and PET quantification measurements in tumor and skin areas (skin/muscle for PET; n=3).

FIGS. 17A-F show ex vivo analysis of tumor section at 24 h after administration of ⁸⁹Zr-SCL.

FIG. 17A shows hematoxylin and eosin staining (expanded regions shown in FIG. 17D and FIG. 17E).

FIG. 17B shows IBA1 immunohistology section (expanded region shown in in FIG. 17F).

FIG. 17C shows autoradiography.

FIG. 17D shows an expanded region of FIG. 17A.

FIG. 17E shows an expanded region of FIG. 17A.

FIG. 17F shows an expanded region of FIG. 17B.

The features and advantages of the present disclosure will become more apparent from the detailed description set forth below when taken in conjunction with the drawings, in which like reference characters identify corresponding elements throughout. In the drawings, like reference numbers generally indicate identical, functionally similar, and/or structurally similar elements.

### DETAILED DESCRIPTION

Throughout the description, where compositions are described as having, including, or comprising specific components, or where methods are described as having, including, or comprising specific steps, it is contemplated that, additionally, there are compositions of the present invention that consist essentially of, or consist of, the recited components, and that there are methods according to the present invention that consist essentially of, or consist of, the recited processing steps.

It should be understood that the order of steps or order for performing certain action is immaterial so long as the invention remains operable. Moreover, two or more steps or actions may be conducted simultaneously.

The mention herein of any publication, for example, in the Background section, is not an admission that the publication serves as prior art with respect to any of the claims presented herein. The Background section is presented for purposes of clarity and is not meant as a description of prior art with respect to any claim.

Described herein is a non-invasive quantitative positron emission tomography (PET) nanoreporter technology that allows personalized therapeutic outcome prediction. In a breast cancer mouse model, it was demonstrated that co-injecting Doxil and a Zirconium-89 nanoreporter (⁸⁹Zr-NRep) allowed highly precise doxorubicin (DOX) quantification. Using ⁸⁹Zr-NRep PET imaging revealed remarkable Doxil accumulation heterogeneity independent of tumor size. Moreover, it was demonstrated that mice with greater than 25 mg/kg DOX accumulation in tumors had significantly better growth inhibition and enhanced survival.

Moreover, described herein is a nanoreporter PET imaging technology that allows robust and accurate in vivo determination of Doxil targeting and DOX tumor content. Together, the presented data and analyses demonstrate that DOX tumor accumulation is an important predictor of breast cancer growth inhibition and a prognostic parameter for survival. More generally, nanoreporter PET can evaluate clinically-approved nanotherapeutics without compromising their therapeutic efficacy. The inherently high sensitivity of PET requires only a small amount of nanoreporter and therefore would not have implications on nanotherapy dosing in patients. The nanoreporter technology disclosed herein can also be used in conjunction with other nanoliposomal anti-cancer therapies, applied to other nanoparticle classes, and/or based on a different imaging platform.

### EXPERIMENTAL EXAMPLES

### Example 1

### Exemplary Labeled Liposome Preparation

An exemplary labeled liposome was prepared as follows. A DFO-conjugated lipid was synthesized by reacting DFO-p-NCS and DSPE in DMSO/chloroform at 40 C for 3 days. Liposomes made of DPPC, cholesterol, DSPE-PEG2000 (1.85:1:0.15) and the conjugate DSPE-DFO (0.3 mol %) were prepared by the sonication method. The DFO-bearing liposomes were labeled by incubation with [89Zr]Zr(C₂O₄)² in PBS at 40 C for 4 h and subsequent purification by spin filtration; preliminary biological evaluation was carried out on female NCr nude mice bearing 4T1 breast tumors.

DSPE-DFO was prepared in 85% yield and the sonication method afforded liposomes of 102.7±4.6 nm (PDI: 0.14±0.02, n=6). Radiolabeling was achieved in 80±10% yield (n=7) and radiochemical purity was >99%. The size of the radiolabeled liposomes was measured to be 108.5±4.6 nm (PDI: 0.15±0.02, n=6). The labeled liposomes were demonstrated to be long-circulating, and biodistribution studies revealed high tumor accumulation, peaking at 24 h p.i. (13.7±1.8% ID/g, n=3). Bone uptake was moderate (3.8% and 5.1% ID/g at 24 and 48 h post-injection, respectively). PET/CT imaging results were in agreement with these observations.

### Example 2

### Doxil Nanoreporter ⁸⁹Zr-NRep

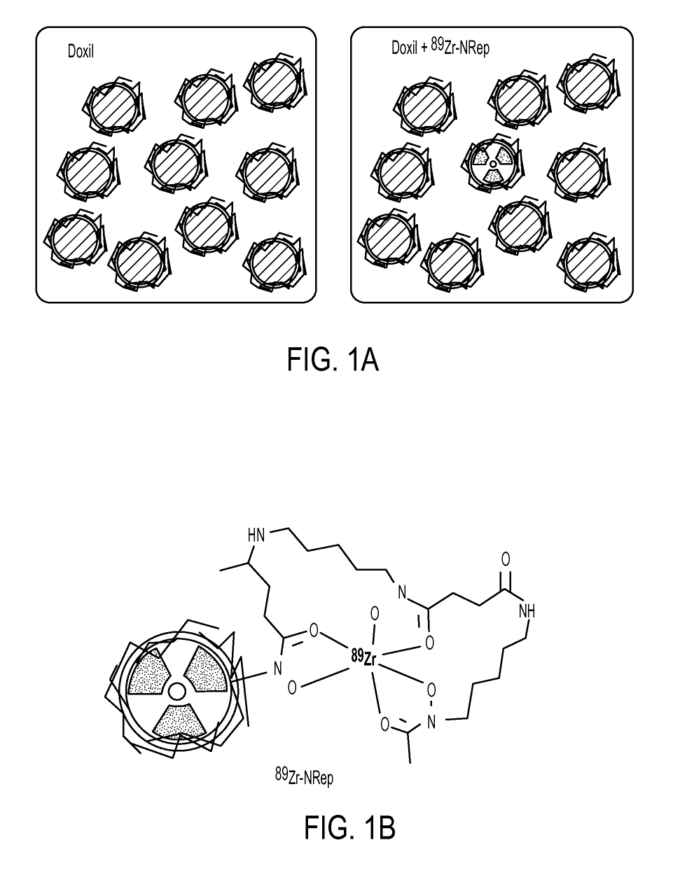

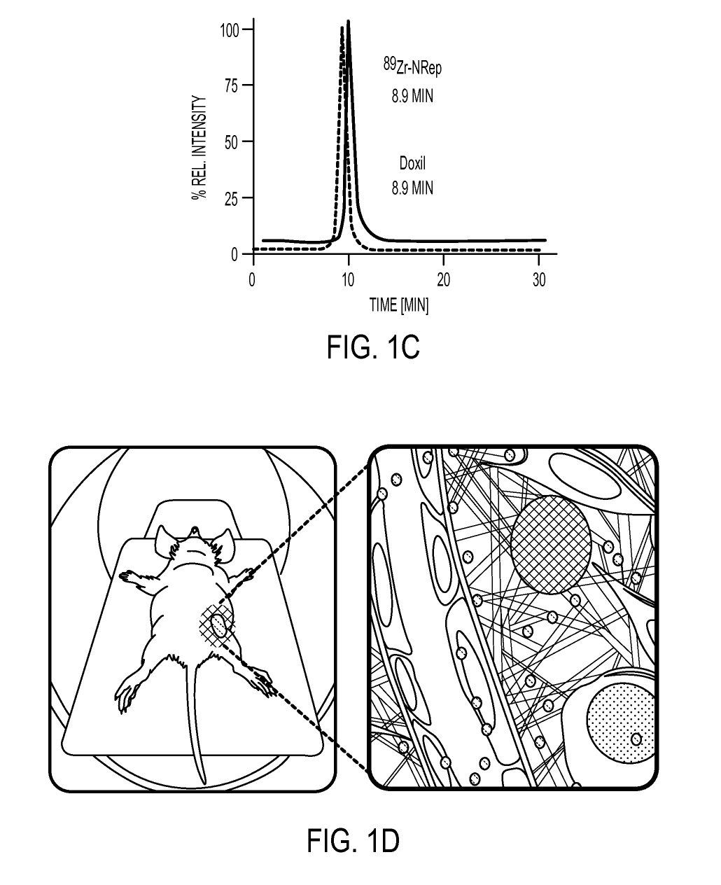

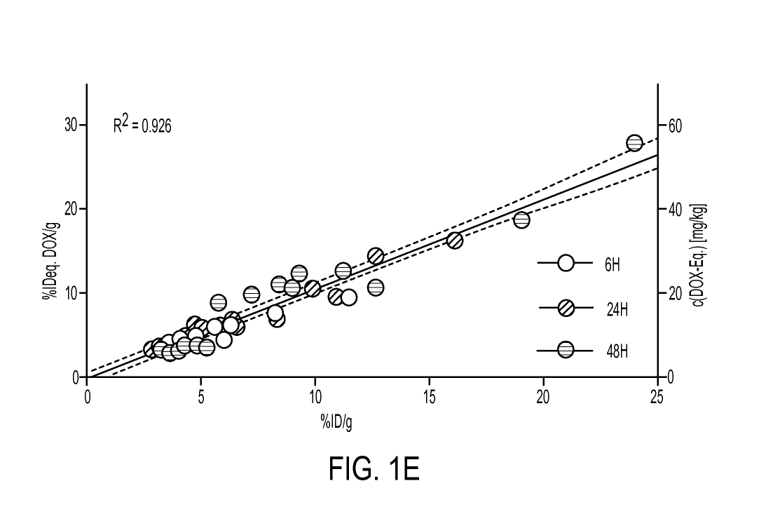

The Doxil nanoreporter ⁸⁹Zr-NRep (FIG. 1A) comprises a pegylated liposome labeled with ⁸⁹Zr through a desferrioxamine B (DFO) functionalized phospholipid (FIG. 1B). The size exclusion chromatography retention time of ⁸⁹Zr-NRep is identical to Doxil (FIG. 1C), and as per dynamic light scattering size measurements, its size and Zeta potential, 100 nm and −20 mV respectively, were very similar to Doxil's (Table 1). Table 1 shows lipid composition (in mol %), size (as mean effective diameter, MED) polydispersity index (PDI) and Z-potential of the different liposomes used.

TABLE 1

| | | Choles- | DSPE- | DSPE- | | | Z-Potential/ |
|---|---|---|---|---|---|---|---|
| | DPPC* | terol | PEG 2000 | DFO | MED/nm | PDI | mV |
| Doxil | 53 | 42 | 5 | — | 82.4 ± 0.2 | 0.05 ± 0.01 | −31.1 ± 11.9 |
| Plain | 61.6 | 33.4 | 5 | — | 103.9 ± 0.7 | 0.11 ± 0.02 | −23.7 ± 4.7 |
| ⁸⁹Zr-NRep | 61.3 | 33.4 | 5 | 0.3 | 113.8 ± 3.1 | 0.15 ± 0.02 | −26.1 ± 8.9** |

*HSPC (hydrogenated soy phosphatidylcholine) for Doxil.
**Unlabeled.

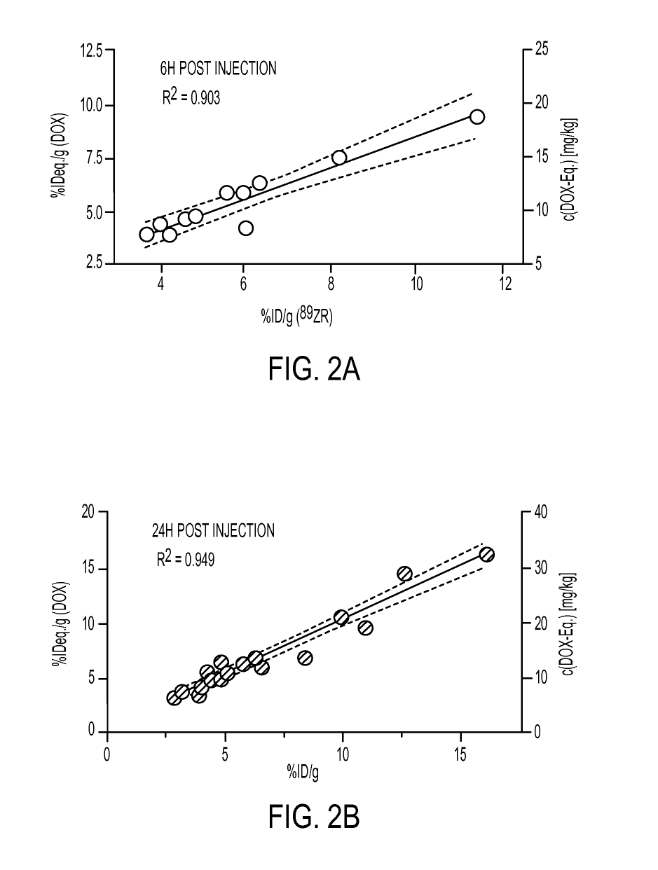

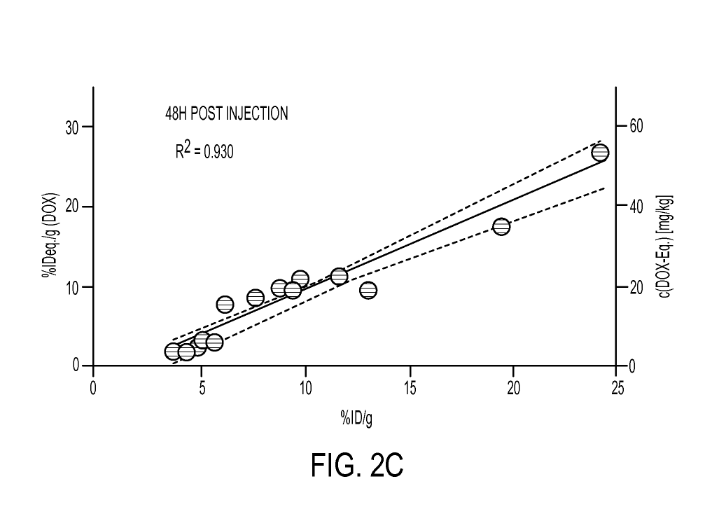

Using a well-established mouse breast cancer model, it was tested whether ⁸⁹Zr-NRep's tumor radioactivity would report on Doxil tumor accumulation as quantified by non-invasive PET imaging (FIG. 1D). To validate the nanoreporter method disclosed herein, a therapeutic dose of Doxil (10 mg/kg) and ⁸⁹Zr-NRep (1.0 mCi/kg) was intravenously co-injected in mice (N=24), and ex vivo quantified radioactivity and DOX content in tumors was measured at 6, 24 and 48 hours after administration (Table 2). A strong correlation (R²=0.93) between the DOX and ⁸⁹Zr-NRep percentages of injected dose per gram tissue (% ID/g) (FIGS. 1E and 2A-2C) was quantified in digested tumor tissue by spectrofluorimetry and gamma counting. The near one slope of this correlation signifies that % ID/g of ⁸⁹Zr equals % ID/g DOX, which indicates that the ratio of Doxil to ⁸⁹Zr-NRep at time of injection remains the same after tumor accumulation.

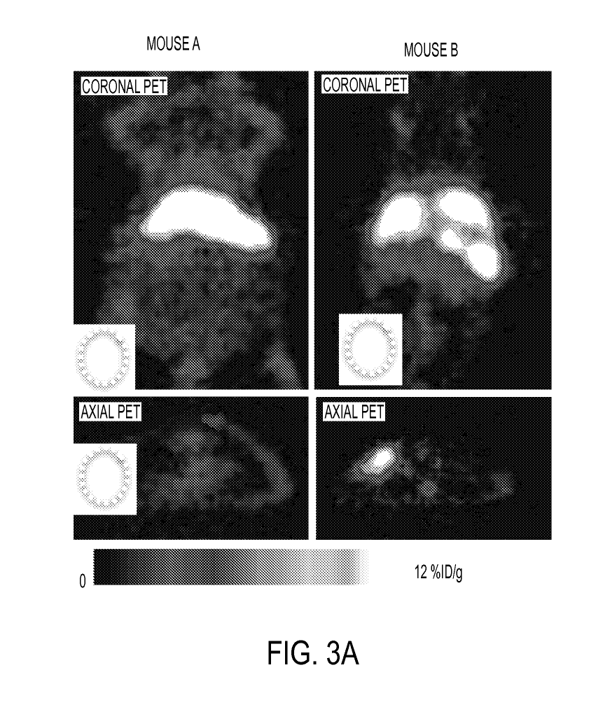

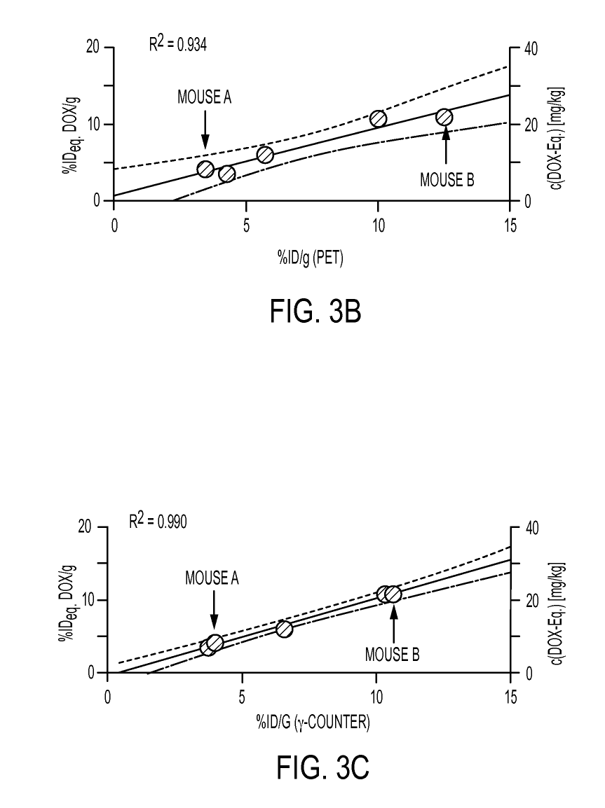

Next, non-invasive PET imaging was used to quantify DOX tumor accumulation. Tumor bearing mice (N=5) were co-administered Doxil (10 mg/kg) and ⁸⁹Zr-NRep (8.0 mCi/kg, Table 2) and underwent in vivo PET imaging 24 hours post injection. FIG. 3A shows two mice with vastly different ⁸⁹Zr-NRep tumor uptake. Following the imaging session the anesthetized mice were euthanized, after which digested tumors' DOX content was determined spectrofluometrically and its radioactivity was quantified by gamma counting. In line with the data presented in FIG. 1E, a strong correlation (R²=0.93) was found between uptake values determined by PET and DOX levels in tumors (FIG. 1B), a result corroborated by ex vivo gamma counting (FIG. 1C).

FIG. 3 shows that mice were injected with ⁸⁹Zr-NRep/Doxil (0.14 mCi ⁸⁹Zr-NRep, 10 mg/kg Doxil), underwent PET imaging at 24 hrs and were then sacrificed to quantify tumor-associated activity measured (γ-counter) and DOX. FIG. 3A shows mouse PET scans, and FIGS. 3B and 3C show the corresponding obtained values.

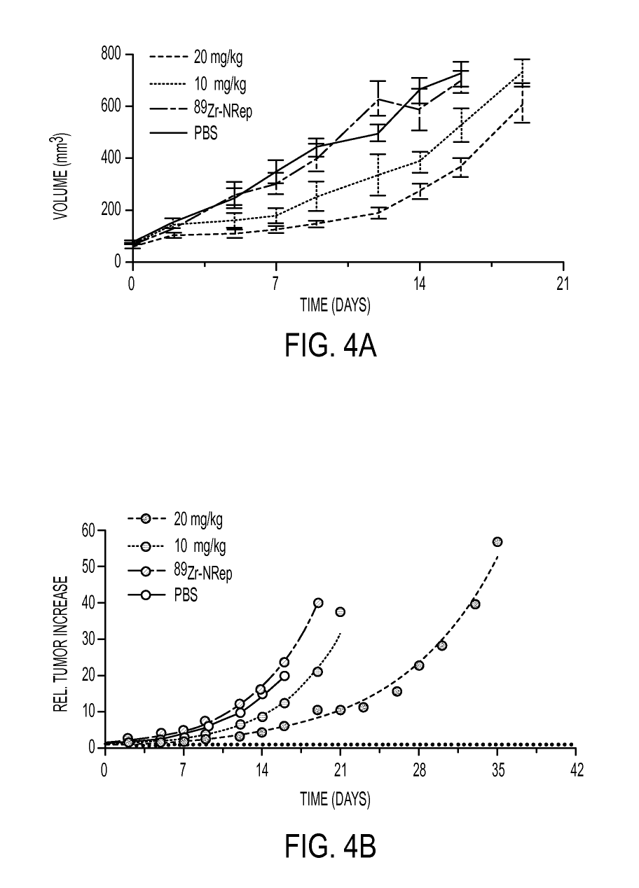

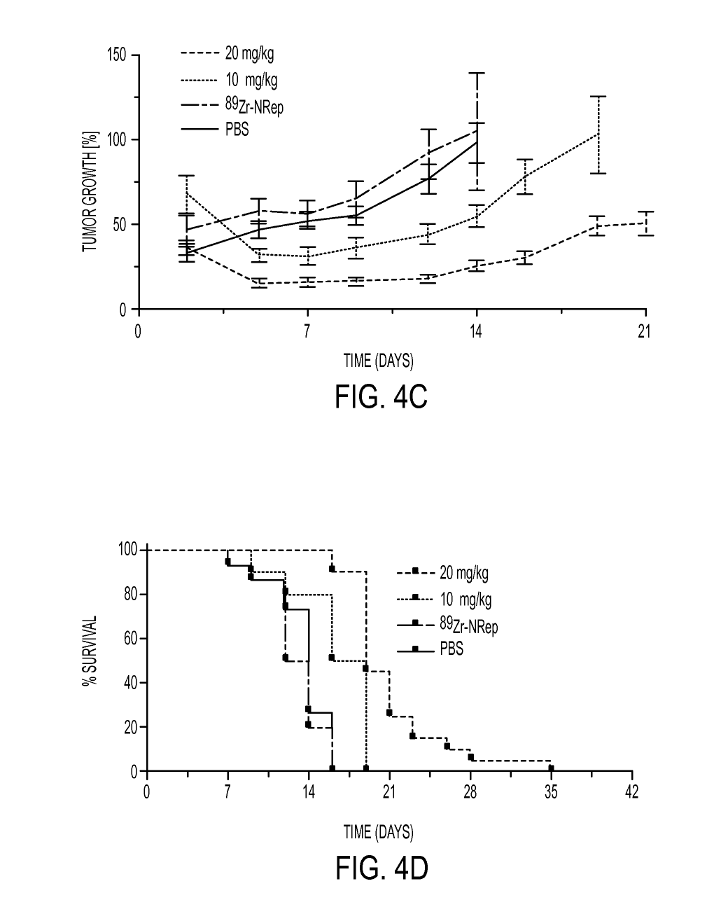

Next, the applicability of the Doxil nanoreporter technology was evaluated for treatment prognosis using an extensive therapeutic characterization. Breast cancer tumor-bearing mice (N=55) were randomly assigned to three different groups: saline control, ⁸⁹Zr-NRep control, and Doxil/⁸⁹Zr-NRep treatment groups dosed at either 10 or 20 mg DOX/kg. At the start of treatment, the tumor sizes among the groups were similar (FIG. 4A). Anesthetized mice from the Doxil/⁸⁹Zr-NRep group underwent a PET imaging session 24 hours post-injection. After this single imaging session, a caliper was used to measure tumor size in all groups three times per week until the animals were euthanized according to defined endpoints. The different groups' tumor growth profiles are shown in FIG. 4A-C. Comparing the ⁸⁹Zr-NRep-injected group and saline-treated controls, it was observed that ⁸⁹Zr-NRep alone did not affect either tumor growth or survival rates (FIGS. 4A-4D). The animals that received Doxil, on the other hand, showed inhibited tumor growth rates and extended survival (FIG. 4C and FIG. 4D). A dose effect was also noted between the high- and low-dose Doxil groups. Finally, by day 12 after treatment administration, the Doxil groups' tumor growth rates approximated those of the untreated groups, which indicated that Doxil's therapeutic effects had worn off (FIG. 4A and 4C).

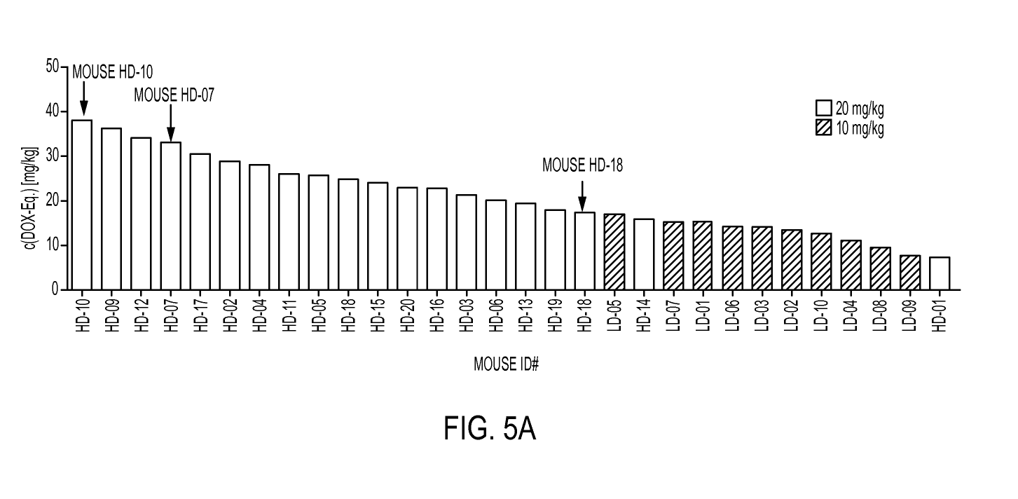

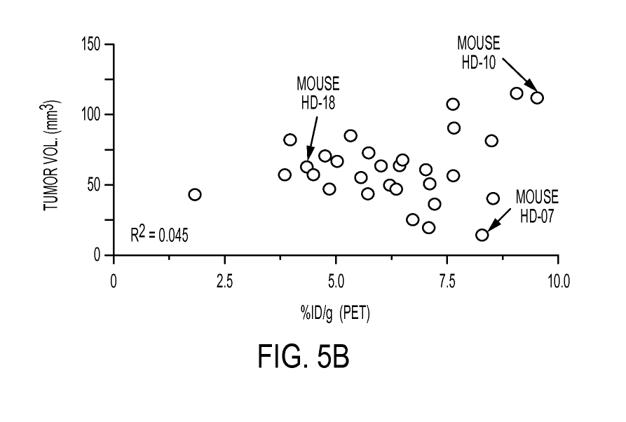

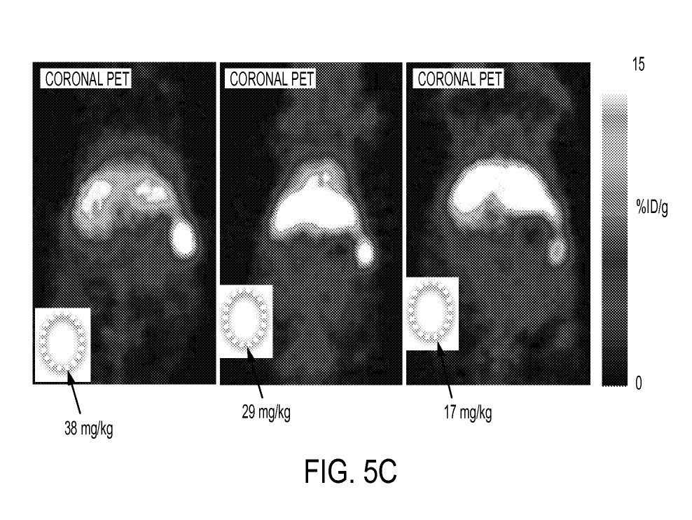

Subsequent analyses of the in vivo ⁸⁹Zr-NRep PET data revealed highly varied DOX accumulation in animals at both Doxil dosage levels (10 and 20 mg/kg). It was found that DOX tumor concentrations ranged from 7.3 mg/kg all the way up to 38.1 mg/kg, indicating that animals receiving the lower dose had lower DOX accumulations in their tumors (FIG. 5A). Moreover, it was also found that no correlation existed between tumor volume and ⁸⁹Zr-NRep uptake (R²=0.05) (FIG. 5B). Thus, without being bound by theory, tumor size does not seem to determine nanotherapeutic penetration. The range of ⁸⁹Zr-NRep uptake heterogeneity is shown in PET images from three different mice (FIG. 5C). FIG. 5C shows high uptake in a large tumor (mouse HD-10), intermediate uptake in a small tumor (mouse HD-17), and low uptake in a large tumor (mouse HD-14).

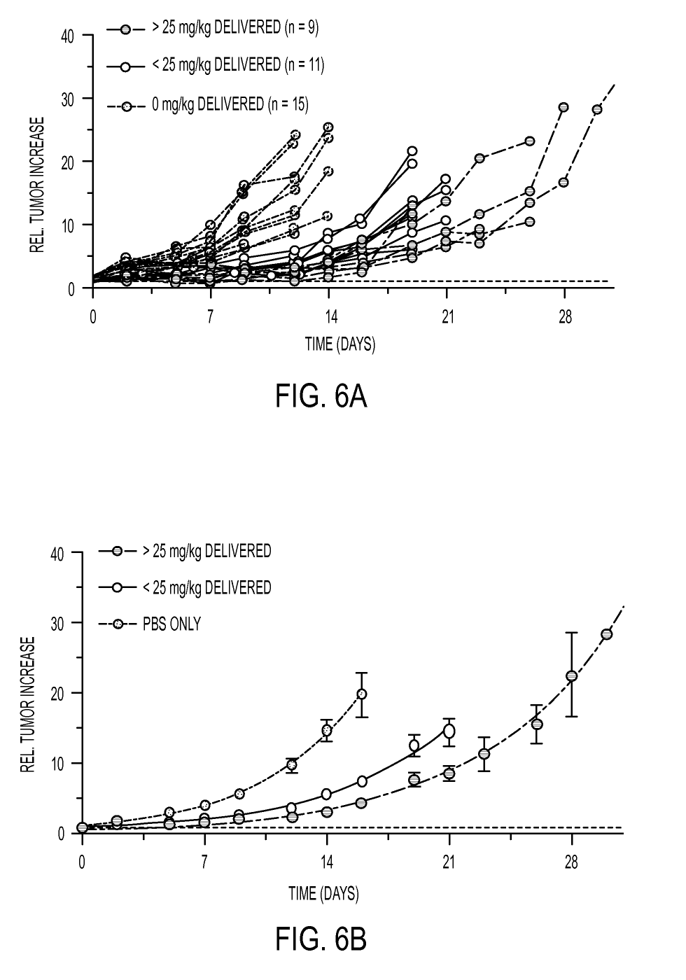

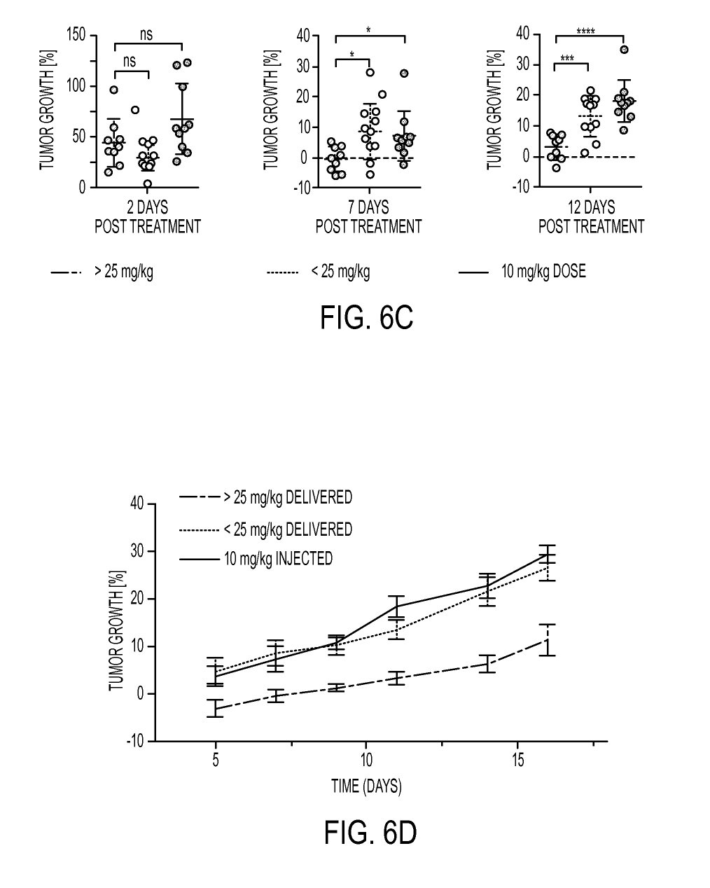

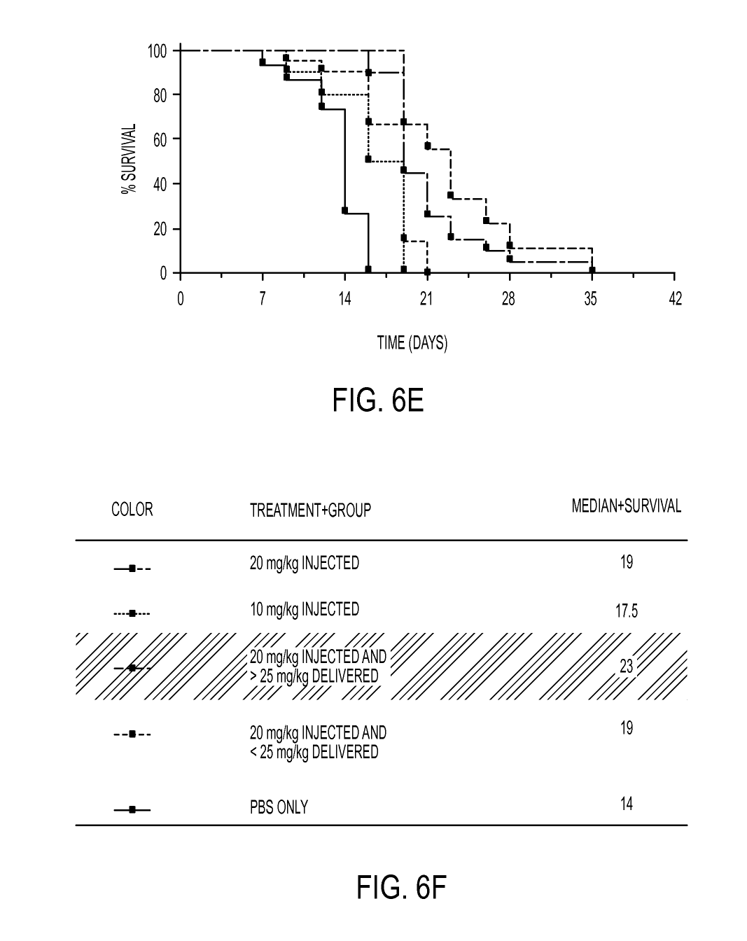

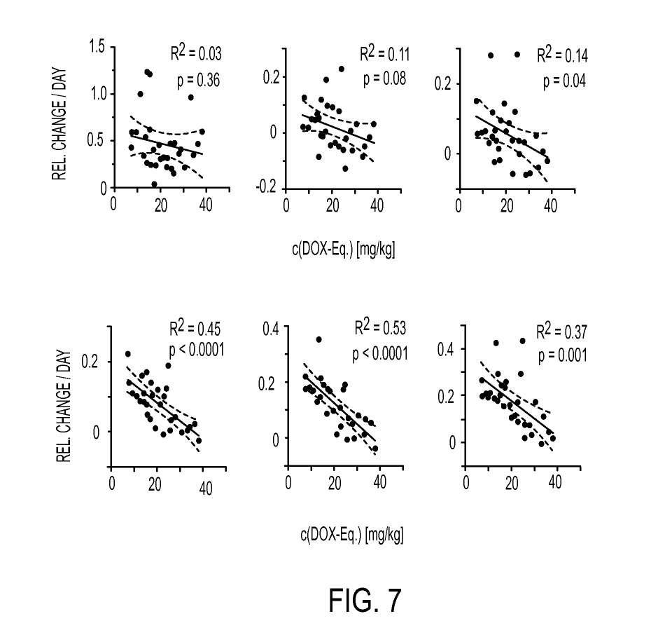

Further investigation into relative tumor growth rates revealed distinct differences between individual mice that received no Doxil and mice that, based on in vivo ⁸⁹Zr-NRep PET, had either less or more than 25 mg/kg DOX accumulation in tumors (FIG. 6A). Based on this observation, the individual animals were subdivided into three groups: controls, less than 25 mg/kg DOX, or greater than 25 mg/kg DOX (FIG. 6B). The group treated with 10 mg/kg Doxil were also included. Two days after Doxil administration and one day after the ⁸⁹Zr-NRep PET scan, no significant differences were seen in percentage tumor growth among the different groups (FIG. 6C). Once therapeutic effects became appreciable (FIG. 7), the average growth rates from that time onwards were determined. At day 7, the greater than 25 mg/kg group had significantly less tumor growth than both the less than 25 mg/kg group (P<0.01) and the animals that received 10 mg/kg Doxil (P<0.02). At day 12 the differences were even more statistically significant (P<0.001 and P<0.0001, respectively; FIGS. 4C and D). All analyses showed the same pattern depicted in FIG. 7 (i.e., the initial lack of correlation changed to a significant correlation by day 12 of treatment). This result indicated that ⁸⁹Zr-NRep PET facilitates tumor growth inhibition and thereby allows retrospective re-categorization using DOX tumor content, measured in vivo, to increase intragroup homogeneity. For individual subjects, the initial ⁸⁹Zr-NRep PET-derived uptake values serve as an inclusion criterion and robust treatment efficacy indicator.

Next, ⁸⁹Zr-NRep PET's prognostic value was investigated. Using the animal subdivisions described above, survival in Kaplan-Meier curves was plotted (FIG. 6E). As with tumor growth, increasingly enhanced survival among control mice, mice treated with 10 mg/kg Doxil, and mice treated with 20 mg/kg Doxil were observed. Median survival for the low- and high-dose Doxil groups was, respectively, 25% and 36% greater than for the control group (FIG. 6F). Subdividing the high-dose Doxil group showed significantly enhanced survival in mice with more than 25 mg/kg DOX accumulated in their tumors. Taking the delivered dose into account, the median survival of animals with more than 25 mg/kg DOX in their tumors is 64% longer than the control group (FIG. 6F).

### Animal Model

The mouse breast cancer cell line 4T1 was obtained from ATCC (Manassas, Va.) and grown in Dulbecco's Modified Eagle's Medium (DMEM) with 4.5 g/L L-glucose, 10% (vol/vol) heat inactivated fetal bovine serum, 100 IU penicillin and 100 μg/mL streptomycin purchased from the culture media preparation facility at Memorial Sloan Kettering Cancer Center (MSKCC, New York, N.Y.). Female homozygous athymic nude NCr mice were obtained from Taconic Laboratories (Hudson, N.Y.). Xenograft injections were performed on mice (8-10 weeks old) anesthetized with 1-2% isoflurane (Baxter Healthcare, Deerfield, Ill.) in 2 L/min medical air. 4T1 cells were injected (1×10⁶ cells in 100 μL DMEM) subcutaneously, and the tumors grown for 7 days.

### Radiosynthesizing ⁸⁹Zr-nanoreporter (⁸⁹Zr-NRep)

Pegylated, chelator-modified liposomes were prepared using the sonication method. For radiolabeling, a solution of 0.3% DFO-bearing liposomes in PBS was reacted with ⁸⁹Zr-oxalate at 40° C. for 2 h. The labeled liposomes were separated from free unreacted ⁸⁹Zr by spin filtration using 100 kDa molecular weight cut-off tubes (Millipore, Billerica, Mass.). The retentate was washed with sterile phosphate buffered saline (PBS, 3×0.5 mL) and finally diluted with sterile PBS to the desired volume. The radiochemical yield was 86±3% (n=6) and the radiochemical purity >99% (FIG. 1C).

### Determining Radioactivity Content and Doxorubicin Concentration in Tumors

Female homozygous athymic nude NCr mice (N=24) bearing 4T1 tumors grown over 7 days were injected with a mixed dose containing Doxil (10 mg doxorubicin/kg body weight) and ⁸⁹Zr-NRep (20.3±3.9 μCi)(Table 2). At predetermined time points (6, 24 and 48 h), animals were sacrificed and perfused with PBS. Tumors were collected and weighed. Larger tumors were divided into portions of approximately 50 mg. The resulting tumor samples were counted using a Wizard² 2480 Automatic Gamma Counter (Perkin Elmer, Waltham, Mass.). Delivered doxorubicin was quantified. Briefly, immediately after gamma counting, tumor samples were homogenized in lysis buffer (10:1 v/w ratio) using a hand-held electrical homogenizer. Aliquots of 200 μL of homogenate were transferred to a new tube, and water (200 μL), Triton X-100 (10:1 dilution in water, 100 μL) and finally acidified isopropanol (0.75 N HCl, 1.5 mL) were added. The mixture was vortex mixed and left at −20 C for 16 h. Samples were then vortexed, and aliquots of 200 μL were measured on a 96 well plate using a Safire microplate reader (Tecan, Männedorf, Switzerland). A calibration curve was generated by adding increasing amounts of doxorubicin to tumor sample homogenates (prepared as described above) from animals not treated with Doxil.

### PET Imaging

Female homozygous athymic nude NCr mice (N=35) bearing 4T1 breast tumors were injected with 0.14-0.17 mCi ⁸⁹Zr-NRep mixed with the corresponding dose of Doxil (see Table 2 for detailed composition). At 24 h the animals were anesthetized with isoflurane/oxygen gas mixture (2% for induction, 1% for maintenance), and a scan was then performed using a Focus 120 microPET scanner (Siemens Medical Solutions, Inc., Malvern, Pa.). Whole body PET static scans recording a minimum of 20 million coincident events were performed, with durations of 10-15 min. The energy and coincidence timing windows were 350-700 keV and 6 ns, respectively. The image data were normalized to correct for non-uniform responses to PET, dead-time count losses, positron branching ratio and physical decay to the time of injection, but no attenuation, scatter or partial-volume averaging correction was applied. The counting rates in the reconstructed images were converted to activity concentrations (percentage injected dose [% ID] per gram of tissue) using a system calibration factor derived from imaging a mouse-sized water-equivalent phantom containing ⁸⁹Zr. Images were analyzed using ASIPro VM™ software (Concorde Microsystems, Knoxville, Tenn.). Activity concentration was quantified at the end of the study by averaging the maximum values in at least 5 ROIs drawn on adjacent slices of tumor tissue.

### Statistical Analysis

Data are expressed as mean±SD or SEM. Data were analyzed using one-way variance analysis (multiple groups) or Student's t-test (two groups) using GraphPad Prism®, Version 6.0c (La Jolla, Calif.), and P-values <0.05 were considered significant.

### Chemicals

Phospholipids were purchased from Avanti Polar Lipids (Alabaster, Ala.). 1-(4-Isothiocyanatophenyl)-3-[6,17-dihyroxy-7,10,18,21-tetraoxo-27-[N-acetylhydroxyla-mino)6,11,17,22-tetraazaheptaeicosane]thiourea (DFO-p-NCS) were purchased from Macrocyclics (Dallas, Tex.). Pegylated liposomal doxorubicin (Doxil) was acquired from the Memorial Hospital pharmacy. All other reagents were acquired from Sigma-Aldrich.

### Synthesizing the Phospholipid-Chelator DSPE-DFO

The phospholipid-chelator DSPE-DFO was prepared. Briefly, 1,2-distearoyl-sn-glycero-3-phosphoethanolamine (DSPE) and 1-(4-Isothiocyana-tophenyl)-3-[6,17-dihyroxy-7,10,18,21-tetraoxo-27-[N-acetylhydro-xylamino)-6,11,17,22-tetraaza-heptaeicosane]thiourea (DFO-p-NCS) were reacted in a 1:1 dimethylsulfoxide/chloroform mixture in the presence of diethyl isopropylaimne at 50° C. for 48 h under nitrogen atmosphere. After cooling down to room temperature, chloroform was evaporated and water was added along with a 1 M Tris solution. The mixture was stirred for 30 min and filtered. The solid was washed with 1 M Tris, water and dichloromethane to produce the desired compound as a white solid in 70-80% yield.

### Liposome Preparation

Liposomes were prepared using a sonication method. Briefly, a lipid film was prepared by evaporating a chloroform solution containing the corresponding lipids in the desired proportion (Table 1). The resulting film was hydrated with PBS (typically 10 mL) and sonicated for 25 min using a 150 V/T Ultrasonic Homogenizer (Biologics, Inc., Ramsey, N.J.) working at 30% power output. After quick centrifugation, size and Z-potential measurements were performed on a NanoSeries Z-Sizer (Malvern Instruments, Malvern, UK) and a Zeta PALS analyser (Brookhaven Instruments Corporation, Holtsville, N.Y.), respectively. Liposomes containing DSPE-DFO were concentrated using a 100 kDa VivaSpin (Millipore, Billerica, Mass.) tube and washed twice with PBS.

### Radiochemistry

⁸⁹Zr was produced at Memorial Sloan-Kettering Cancer Center on an EBCO TR19/9 variable-beam energy cyclotron (Ebco Industries Inc., BC, Canada) via the ⁸⁹Y(p,n)⁸⁹Zr reaction and purified in accordance with previously reported methods to yield ⁸⁹Zr with a specific activity of 195-497 MBq/μg. Activity measurements were made using a Capintec CRC-15R Dose Calibrator (Capintec, Ramsey, N.J.).

### HPLC and Radio-HPLC

HPLC was performed on a Shimadzu HPLC system equipped with two LC-10AT pumps and an SPD-M10AVP photodiode array detector. Radio-HPLC was performed using a Lablogic Scan-RAM Radio-TLC/HPLC detector. Analytical runs were carried out on either C18 Waters Atlantis T3 column (6×250 mm, 5 μm) or a C4 Phenomenex Jupiter column (4.6×250 mm, 5 μm). The solvent systems used were water (0.1% TFA, solvent A), acetonitrile (0.1% TFA, solvent B) and methanol/acetonitrile 60:40 (0.1% TFA, solvent C) with a flow rate of 1 mL/min. Size exclusion chromatography was performed on a Superdex 10/300 column (GE Healthcare Life Sciences) using PBS as eluent at a flow rate 1 mL/min.

### Animal Care

For all intravenous injections, mice were gently warmed with a heat lamp and placed on a restrainer. Their tails were sterilized with alcohol pads, and injections were placed into the lateral tail vein. All animal experiments were done in accordance with protocols approved by the Institutional Animal Care and Use Committee of MSKCC following National Institutes of Health guidelines for animal welfare.

### Therapeutic Study

Female homozygous athymic nude NCr mice (8-10 weeks old, n=55) bearing 4T1 breast tumors grown over 7 days were divided into three groups receiving saline (PBS, N=15), ⁸⁹Zr-NRep (N=10) and Doxil/⁸⁹Zr-NRep (N=30). The last group was divided in two subgroups according to the DOX dose injected (10 or 20 mg/kg, N=10 and N=20, respectively). Dose composition can be found in Table 2. Table 2 shows composition (expressed as μmol of total lipids, activity and mg of doxorubicin), size (MED) and polydispersity index (PDI) of the doses described herein. The doses were administered via the lateral tail vein. The treatment groups (low and high DOX) had a PET imaging scan 24 h post injection. Control groups were anesthetized for the duration of a typical PET scan (10-15 min) as a mock imaging session. All groups were monitored for tumor size three times weekly using digital calipers to take the longest (L) and shortest (S) perpendicular diameters. The volume was calculated using the formula V=(L×S2)/2. Euthanasia was scheduled according to predetermined endpoints: either a tumor volume greater than 600 mm³ or notification by the Research Animal Resource Center (RARC) personnel from Memorial Sloan Kettering Cancer Center. After the end of the study, PET images were analyzed as described above to determine uptake values.

TABLE 2

| | | | ⁸⁹Zr-NRep/ | | | |
|---|---|---|---|---|---|---|
| | Doxil | Plain | μCi | DOX | MED/nm | PDI |
| Ex vivo | 2.4 μmol | — | 20.3 ± 3.9 | 0.2 mg | 83.0 ± 1.2 | 0.06 ± 0.01 |
| In vivo | 2.4 μmol | — | 142 ± 1 | 0.2 mg | 88.9 ± 0.7 | 0.10 ± 0.03 |
| ⁸⁹Zr-NRep only | — | 4.8 μmol | 163 ± 16 | — | 106.7 ± 1.6 | 0.12 ± 0.01 |
| Low Doxil | 2.4 μmol | 2.4 μmol | 163 ± 13 | 0.2 mg | 94.1 ± 1.6 | 0.11 ± 0.01 |
| High Doxil | 4.8 μmol | — | 165 ± 12 | 0.4 mg | 87.4 ± 1.2 | 0.09 ± 0.01 |

### Example 3

### ⁸⁹ Zr-Labeled Liposomes Prepared Using Two Different Approaches-Click Labeling and Surface Chelation

Pharmacokinetic and biodistribution studies, as well as PET/CT imaging of the radiolabeled nanoparticles were performed in a mouse model of breast cancer. In addition, a dual PET/optical probe was prepared by incorporation of a near-infrared fluorophore and tested in vivo by PET and near-infrared fluorescence imaging. The surface chelation approach proved to be superior in terms of radiochemical yield and stability, as well as in vivo performance. Accumulation of these liposomes in tumor peaked at 24 hours post injection and was measured to be 13.7±1.8% ID/g. The in vivo performance of this probe was not essentially perturbed by the incorporation of a near infrared fluorophore. In xenograft and orthotopic mouse models of breast cancer, their biodistribution was visualized by PET imaging. In combination with a near infrared dye, these liposomal nanoparticles can serve as bimodal PET/Optical imaging agents. It was shown that the liposomes target malignant growth and that their bimodal features may be useful for simultaneous PET and intraoperative imaging.

### Synthesis of Functional Lipids

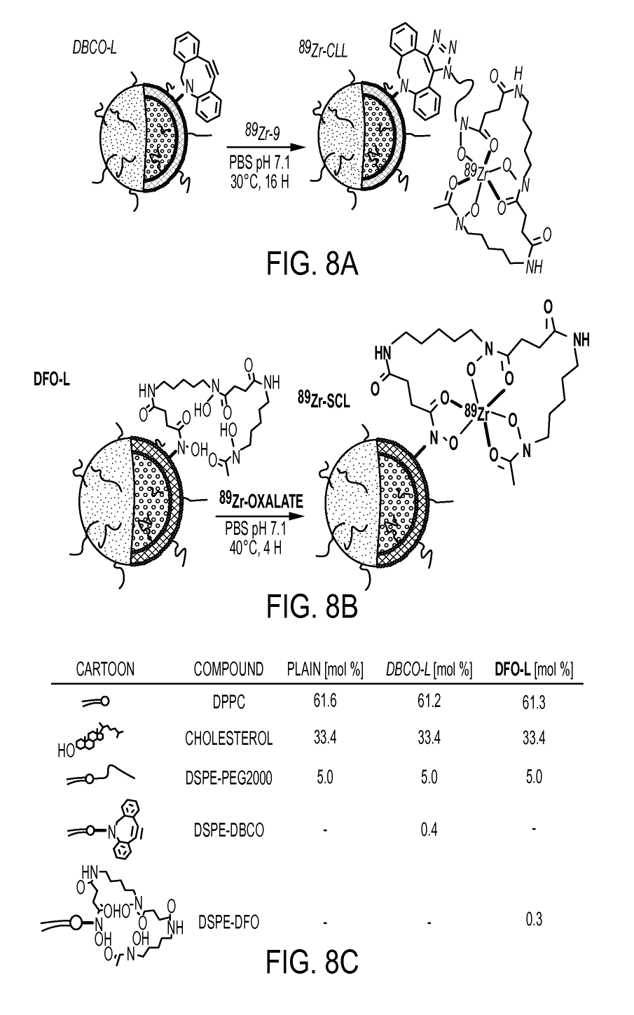

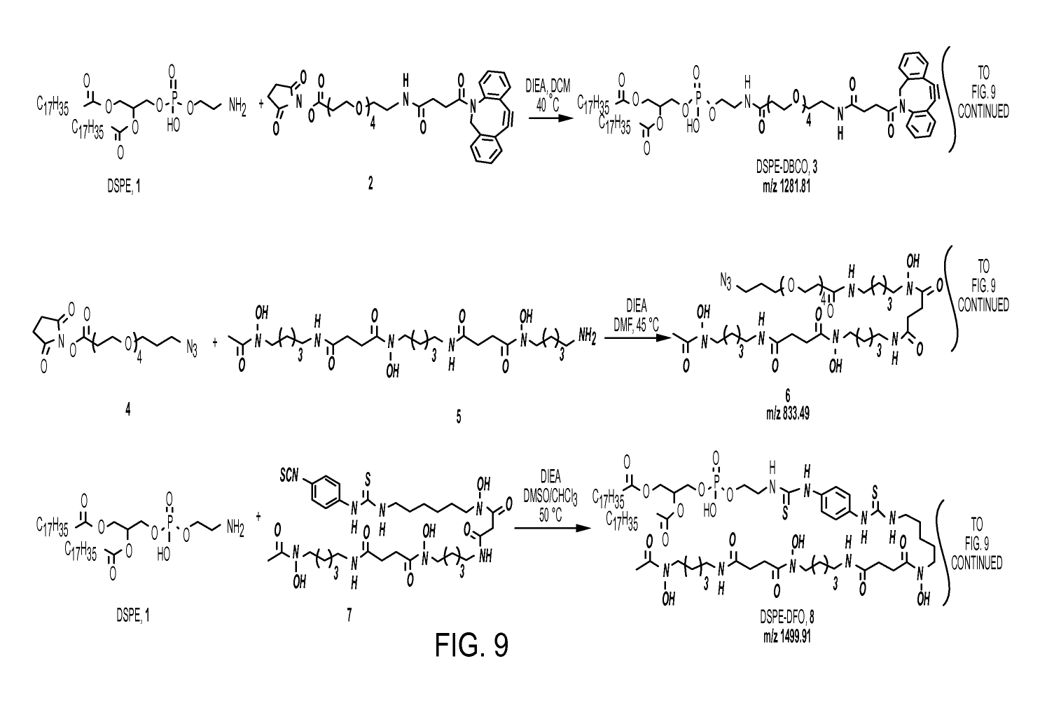

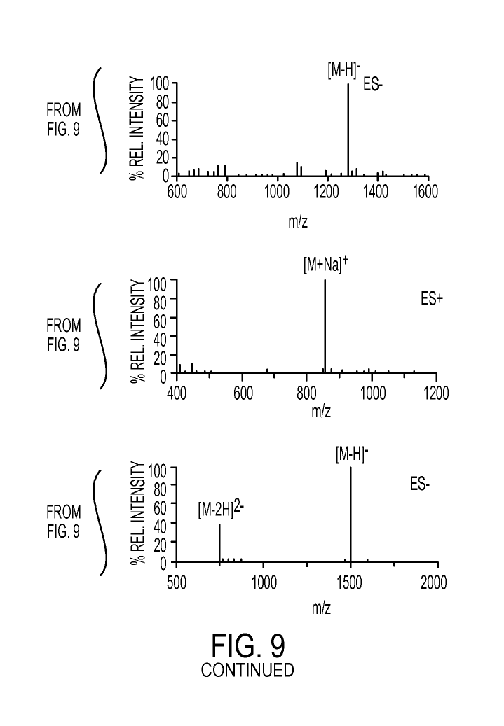

The formulation of DBCO-L and DFO-L (FIGS. 8A and 8B, respectively) required the synthesis of two derivatives of the phospholipid 1,2-distearoyl-sn-glycero-3-phosphoethanolamine (DSPE, 1, FIG. 9). Reaction of DSPE with the pegylated dibenzo-azacyclooctyne (DBCO)-NHS ester 2 and the desferrioxamine (DFO)-p-benzoisothiocyante reagent 7 furnished compounds 3 and 8 in good yield (57 and 85%, respectively). Additionally, a clickable DFO-azide construct (6) was prepared using desferrioxamine mesylate 5 and the pegylated azide NHS ester 4 (FIG. 9).

### Formulation of Liposomal Nanoparticles

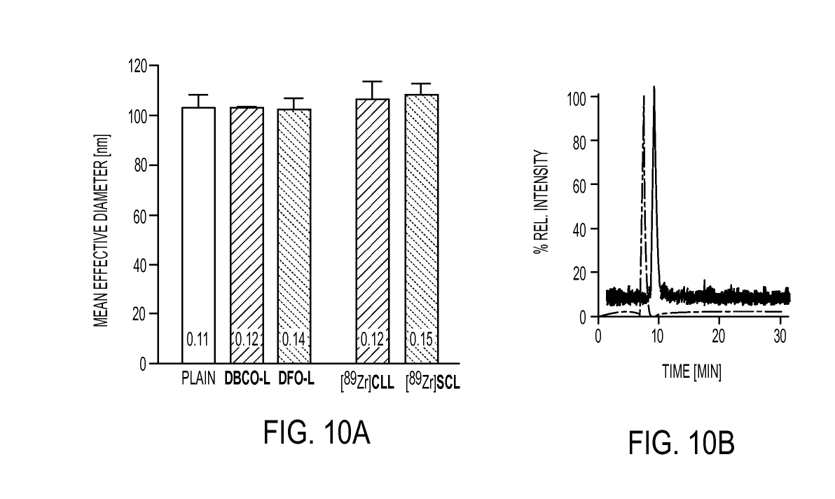

Non-functionalized liposomes composed of 1,2-dipalmitoyl-sn-glycero-3-phosphocholine (DPPC), cholesterol and pegylated DSPE (DSPE-PEG2000) in a 1.85:1:0.15 mole ratio were prepared by adding the individual lipids to a reaction vessel in a solution of chloroform, removing the organic solvent, and adding phosphate-buffered saline (PBS), followed by sonication and centrifugation. This procedure yielded liposomal nanoparticles with a mean diameter of 100 nm and a low polydispersity (mean effective diameter, MED: 103.4±5.1 nm, PO1: 0.11±0.01, n=4), with a slightly negatively charged surface (Z-potential: −21.7±5.4 mV, n=3). The functionalized counterparts of the non-labeled liposomes, DBCO-L and DFO-L, were prepared identically, but had the lipids 3 and 8 added to their respective initial lipid mixtures (0.4% for DBCO-L and 0.3% for DFO-L), at the expense of DPPC (FIG. 8C). The presence of the new lipids was well tolerated, and size as well as polydispersity of the resulting liposomes were comparable to that of the plain liposomes (MEDoFo-L: 102.7±4.6 nm, PDioFo-L: 0.14±0.02, n=6; MEDosco-L: 102.3±1.4 nm, PDiosco-L: 0.12±0.01, n=3, FIG. 10A). Both preparations were stable for weeks at 4° C. with no measurable aggregation or loss in reactivity. The surface charge was not substantially affected either, although a slight variation towards neutrality was observed for DBCO-L (Z-potential DFO-L: −22.6±3.9 mV, n=4, DBCO-L: −15.9±3.1 mV, n=2).

### Radiolabeling and Stability of Liposomes

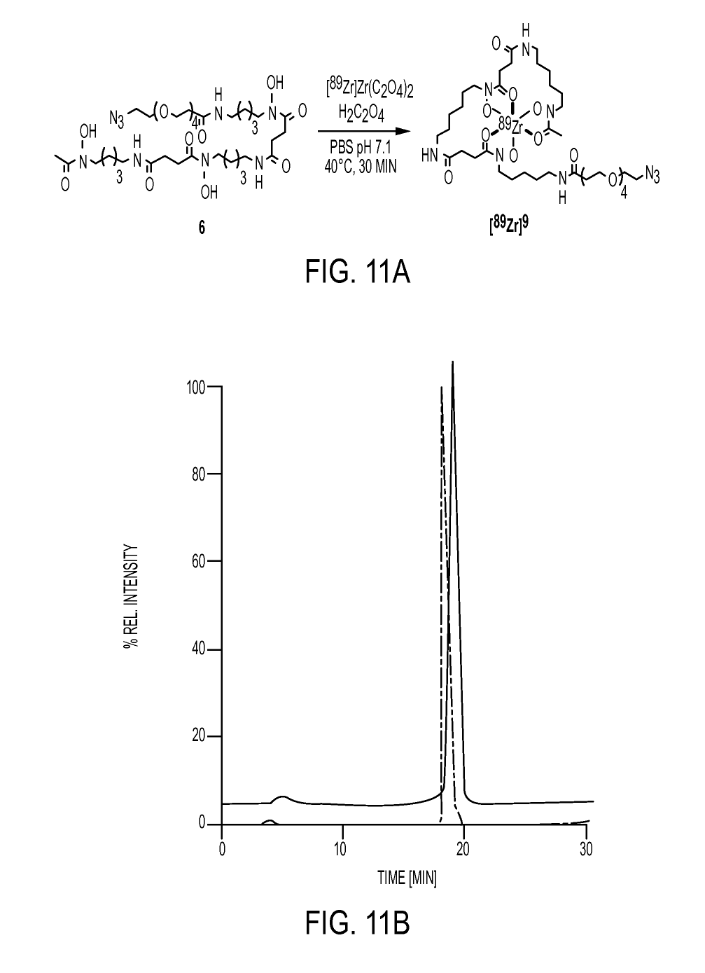

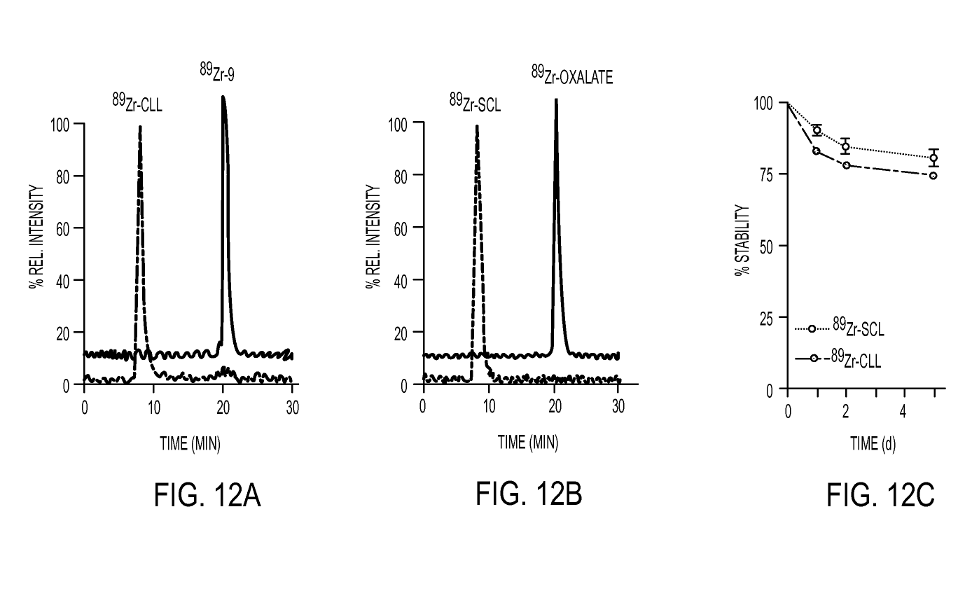

The preparation of click-labeled liposomes (⁸⁹Zr-CLL, FIG. 8A) required the labeling of the bioorthogonally reactive DFO derivative 6 with ⁸⁹Zr, which was obtained in 44±15% yield after isolation by HPLC (FIGS. 11A-11B). The isolated labeled fragment, ⁸⁹Zr-9, was then reacted with DBCO-L overnight at 30° C. (FIG. 8A). Purification of ⁸⁹Zr-CLL by spin filtration afforded the radiolabeled material in 14±4% radiochemical yield (RCY) and >95% purity (FIG. 12A). The labeling process did not affect the distribution (size and PDI) of the sample as compared to DBCO-L (MEDaszr-CLL: 106.4±7.3, PDiaszr-CLL: 0.12±0.02, n=3, FIG. 10A). In a control experiment, non-functionalized liposomes did not take up nor adsorb any radioactivity when incubated in the same conditions with ⁸⁹Zr-9. When ⁸⁹Zr-CLL was disassembled after the addition of ethanol, the radiolabeled clicked lipid by radio-HPLC was able to be detected, confirming successful biorthogonal ligation.

The radiolabeling of liposomes by surface chelation was achieved by incubation of DFO-L (FIG. 8B) with a ⁸⁹Zr-oxalate solution in PBS at pH 7.1-7.4 for 4 hours at 40° C. and purified by spin filtration. The RCY was 80±10% (n=7) and the resulting liposomes, ⁸⁹Zr-SCL, were radiochemically pure (FIG. 12B). No statistically significant increase in the size of the liposomes was observed after labeling, compared to precursor DFO-L (MED⁸⁹Zr-SCL: 108.5±4.6, PDI⁸⁹Zr-SCL: 0.15±0.02, n=6, FIG. 10A). A control experiment was carried out to assess the binding of ⁸⁹Zr to the surface of plain, non-functionalized liposomes. Incubation of a sample of these liposomes with ⁸⁹Zr-oxalate at 40° C. over a period of 16 hours afforded less than 0.5% of activity bound to liposomes. The radiolabeled lipid was also detected by radio-HPLC analysis of a sample of disassembled liposomes.

The stability of both labeled liposome preparations was assessed in serum (FBS) by incubation of samples at 37° C. for a period of five days (FIG. 12C). Release of activity (10% for ⁸⁹Zr-SCL and 17% for ⁸⁹Zr-CLL, after 24 hours) was observed for both labeling strategies. At later time points, a fraction of plasma proteins with an estimated molecular weight of 40 kDa took up some of the liberated activity (less than 3% and less than 6% after 48 h and 120 h, respectively). However, it remains challenging to establish whether this effect is based on the chelation of free ⁸⁹Zr by the plasma protein fraction or to lipid exchange between the liposomes and plasma proteins. Overall, however, these data indicate a high stability of the chelator on the lipid surface of both ⁸⁹Zr-SCL and ⁸⁹Zr-CLL (FIG. 12C).

Pharmacokinetics and In Vivo Imaging with ⁸⁹Zr-Labeled Liposomes

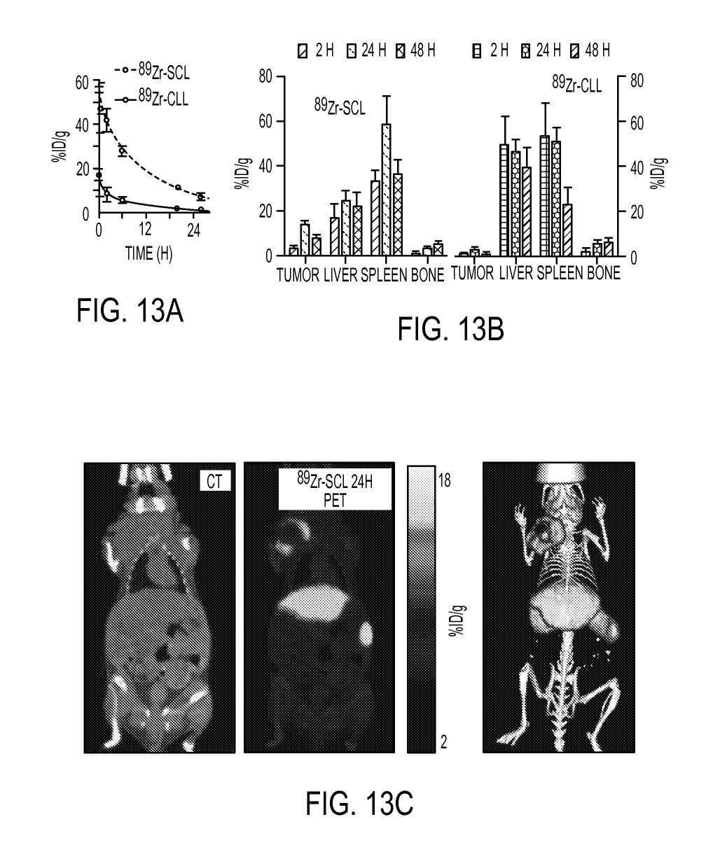

In vivo evaluation of both labeled liposomes started with the measurement of their blood half-life in healthy female NCr nude mice (FIG. 13A). The weighted half-life for ⁸⁹Zr-SCL proved to be markedly longer than that of ⁸⁹Zr-CLL (t_{1/2}=7.20 h and 1.25 h, respectively). FIG. 13B shows a comparative ⁸⁹Zr activity biodistribution in selected tissues after intravenous administration of the liposomes in mice bearing 4T1 breast cancer xenografts, which was chosen as a representative model of solid tumors (Table 3 and Table 4).

Table 3 shows the tissue radioactivity distribution of 89Zr-SCL in female NCr nude mice bearing 4T1 breast xenografts (n greater than or equal for each time point).

TABLE 3

| | 2 h | | 24 h | | 48 h | |
|---|---|---|---|---|---|---|
| Tissue | % ID/g | SD | % ID/g | SD | % ID/g | SD |
| Blood | 36.9 | 1.65 | 6.81 | 0.29 | 1.89 | 0.76 |
| Tumor | 3.29 | 1.11 | 13.7 | 1.84 | 7.88 | 1.16 |
| Heart | 0.97 | 0.19 | 1.14 | 0.14 | 1.26 | 0.07 |
| Lungs | 1.12 | 0.35 | 1.05 | 0.22 | 1.03 | 0.15 |
| Stomach | 1.30 | 0.51 | 1.57 | 0.39 | 0.76 | 0.07 |
| Small intestine | 2.62 | 1.07 | 2.72 | 0.49 | 1.41 | 0.10 |
| Large intestine | 1.95 | 0.97 | 1.56 | 0.23 | 0.93 | 0.05 |
| Spleen | 33.5 | 4.72 | 58.9 | 12.2 | 36.0 | 7.03 |
| Kidneys | 2.64 | 0.50 | 3.36 | 0.43 | 3.19 | 0.21 |
| Liver | 17.1 | 6.22 | 24.7 | 4.59 | 22.2 | 6.15 |
| Muscle | 0.85 | 0.01 | 1.13 | 0.09 | 1.77 | 0.78 |
| Bone | 1.50 | 0.32 | 3.78 | 0.08 | 5.09 | 1.32 |

Table 4 shows tissue radioactivity distribution of of ⁸⁹Zr-CLL in female NCr nude mice bearing 4T1 breast xenografts (n is greater than or equal to 3 for each time point).

TABLE 4

| | 2 h | | 24 h | | 48 h | |
|---|---|---|---|---|---|---|
| Tissue | % ID/g | SD | % ID/g | SD | % ID/g | SD |
| Blood | 5.53 | 0.35 | 1.04 | 0.20 | 0.68 | 0.24 |
| Tumor | 1.23 | 0.26 | 2.00 | 0.15 | 1.73 | 0.19 |
| Heart | 0.80 | 0.06 | 0.82 | 0.23 | 0.67 | 0.26 |
| Lungs | 1.19 | 0.06 | 0.72 | 0.16 | 0.71 | 0.28 |
| Stomach | 0.59 | 0.15 | 0.42 | 0.05 | 0.40 | 0.10 |
| Small intestine | 0.51 | 0.09 | 0.27 | 0.04 | 0.28 | 0.05 |
| Large intestine | 0.52 | 0.12 | 0.27 | 0.03 | 0.22 | 0.10 |
| Spleen | 54.3 | 14.3 | 51.4 | 6.14 | 23.3 | 7.29 |
| Kidneys | 2.40 | 0.65 | 2.75 | 0.17 | 3.24 | 0.40 |
| Liver | 49.7 | 13.0 | 46.7 | 5.49 | 40.1 | 8.34 |
| Muscle | 1.42 | 0.24 | 1.19 | 0.35 | 0.99 | 0.47 |
| Bone | 2.59 | 0.92 | 5.53 | 1.57 | 6.16 | 1.83 |

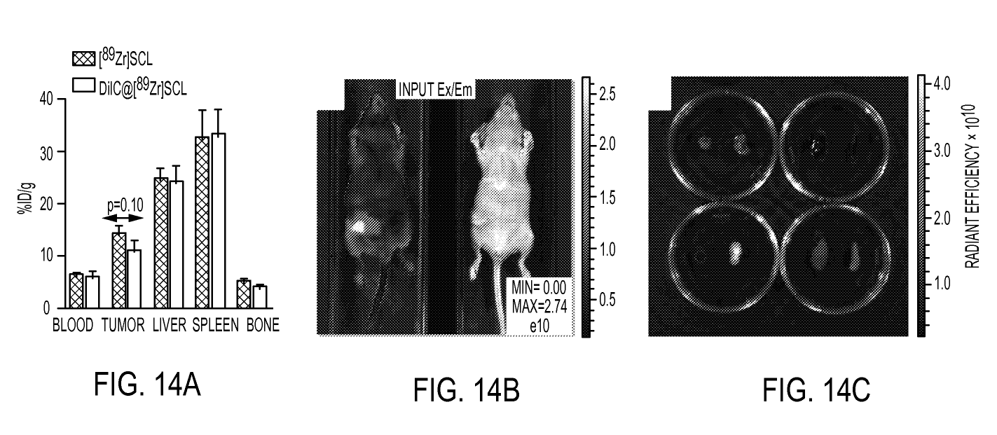

PET/CT imaging with ⁸⁹Zr-CLL at 2 h post injection shows predominantly liver and spleen uptake. There was no substantial difference at 24 h post injection (FIGS. 14A-14C) and subsequent time points. In contrast, ⁸⁹Zr-SCL PET images at first showed high blood pool activity (24.3±4.2 percentage injected dose per gram of tissue (% ID/g) in heart, n=2) and also strong signals from liver and spleen. At 24 h, the blood pool signal was moderate (6.2±0.4% ID/g in heart, n=4), but tumor accumulation was considerably higher. In accordance with the biodistribution results, spleen and liver tissues showed highest uptake/accumulation at all time points. Quantitative data obtained from PET scans were in good agreement with the biodistribution results. FIG. 13C shows that the overall tumor uptake at 24 h was high, and measured 14.1±1.6 (n=4) % ID/g. Later time points show blood activity clearance but persistent PET signal in tumor (12.7±1.0 and 10.2±0.5% ID/g, n=2, at 48 hand 120 h, respectively).

### Histology

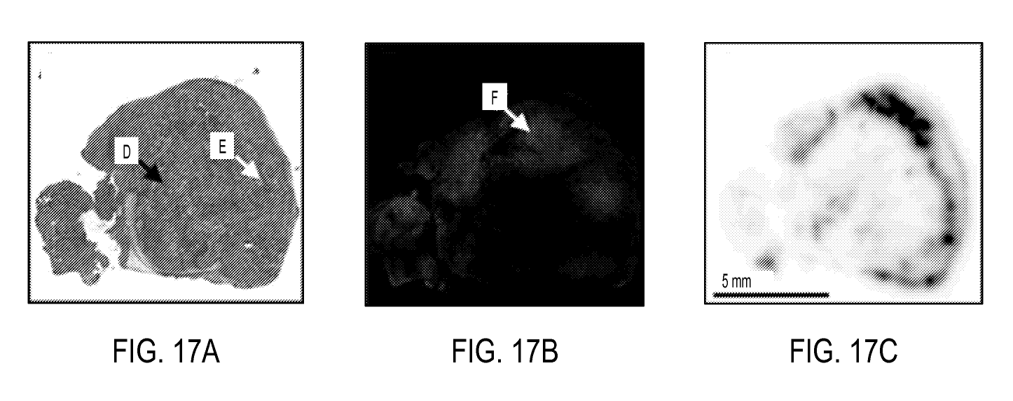

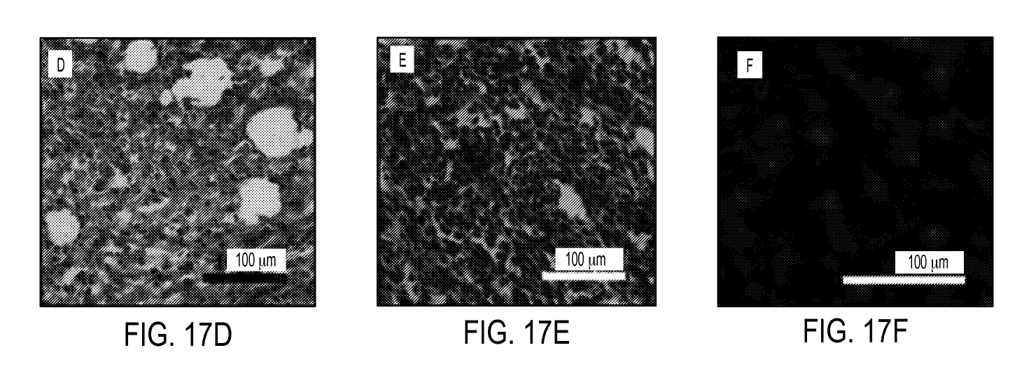

Ex vivo analysis by autoradiography and histological staining of tumor sections (excised at 24 h after administration of ⁸⁹Zr-SCL) was performed to elucidate the regional distribution of the radiotracer (FIG. 17). Hematoxylin and eosin staining (FIG. 17A) revealed an inner region characterized by reduced number of nuclei and viable cells, which, in some embodiments, may develop with necrotic core (FIG. 17D), as opposed to the external areas, which show higher cell-density and normal appearance (FIG. 17E). The inner region did not stain for IBA-1 (ionized calcium binding adaptor molecule 1, FIG. 17B), which is specifically expressed in macrophages and microglia, and has low accumulation of ⁸⁹Zr-SCL (FIG. 17C).

Bimodal Imaging with Cy5/⁸⁹Zr Labeled Liposomes

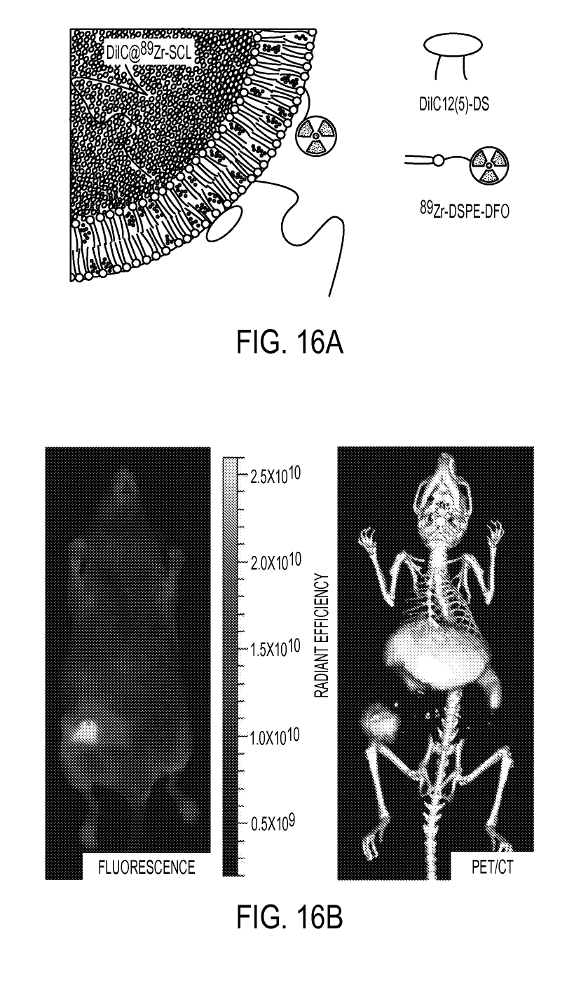

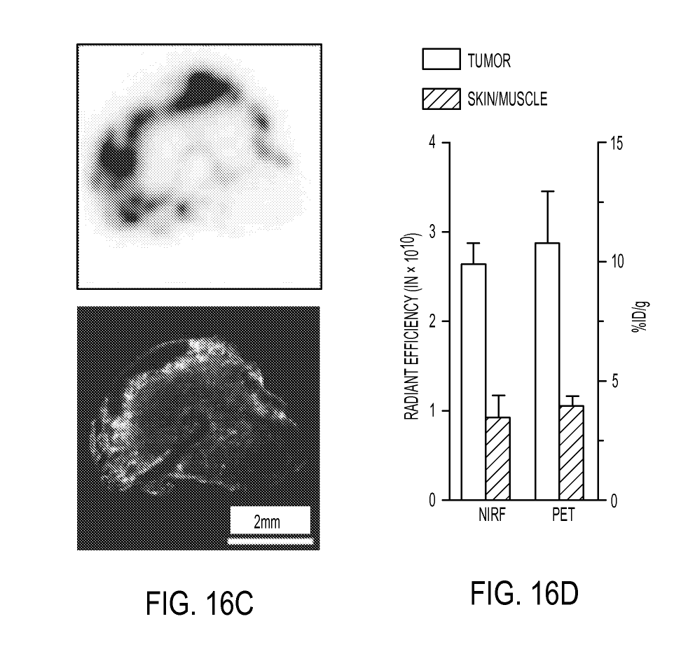

A ready-to-label liposome incorporating the NIR dye DilC12(5)-0S, namely DilC@OFO-L was labeled with ⁸⁹Zr following the same procedure used for ⁸⁹Zr-SCL yielding the bimodal liposome DilC@⁸⁹Zr-SCL (FIG. 16A) in 88% RCY and greater than 99% radiochemical purity (FIG. 10B). Subsequent PET imaging showed that the fluorescent DilC@89Zr-SCL was also long-circulating and had essentially the same performance as ⁸⁹Zr-SCL (FIG. 14A). Histological analysis of tumor sections revealed a high level of co-localization of radioactivity and fluorescence, as shown in FIG. 16C. Both signals are localized to the periphery of the tissue, indicating the progress of an incipient necrotic core. Furthermore, same-animal analysis (n=3) of PET/CT and epifluorescence NIR imaging at 24 h post-injection (FIG. 16B) yielded an excellent correlation between the two imaging modalities (FIG. 16D) on the whole body level. The tumor-to-skin(fluorescence)/skin-muscle(PET) ratio was measured to be 2.8 and 2.9 using PET/CT and NIRF data, respectively, and compares well with the values obtained from biodistribution experiments (tumor/skin=2.3).

### Materials and Equipment

Phospholipids were purchased from Avanti Polar Lipids, whereas compounds 2 and 5 were supplied by Click Chemistry Tools, and compound 7 by Macrocyclics. The dye DilC12(5)-DS [1,1-Diododecyl-3,3,3,3-tetramethyl-indodicarbocyanine-5,5-disulfonic acid] was purchased from AAT Bioquest. All other reagents were purchased from Sigma-Aldrich. All chemicals were used without further purification. Column chromatography was performed on silica-gel (Silicycle, 40-63 μm, 230-400 mesh). 1H-NMR spectra were recorded at room temperature on Bruker Avance 500 instrument operating at the frequency of 500 MHz. All were internally referenced to the residual solvent peaks, CDCb (7.26 ppm), DMSO-d6 (2.49 ppm) or CD30D (3.31 ppm). High-resolution mass data were recorded on a Waters LCT Premier XE mass spectrometer.

### Cell Culture

The mouse breast cancer cell line 4T1 was obtained from ATCC (Manassas, Va.) and grown in Dulbecco's Modified Eagle's Medium (DMEM) with 4.5 g/L L-glucose, 10% (vol/vol) heat inactivated fetal bovine serum, 100 IU penicillin, and 100 ug/ml streptomycin and purchased from the culture media preparation facility at Memorial Sloan Kettering Cancer Center (MSKCC New York, N.Y.).

### Animals

Female homozygous athymic nude NCr mice were obtained from Taconic Laboratories (Hudson, N.Y.). For xenograft injections, mice were anesthetized with 1-2% isoflurane gas in 2 Limin medical air, before 4T1 cells were injected (1×10⁶ cells in 100 μL DMEM) subcutaneously, and the tumors grown for 10-12 days. For orthotopic injections, mice were anesthetized with a 150 mg/kg ketamine and 15 mg/kg xylazine cocktail (10 μL) and an incision was made above the mammary fat pad after sterilization of the region. Then, 4T 1 cells (1×106 cells in 100 μL DMEM) were injected into the mammary fat pad, before the incision was sealed (Vetbond, 3M, St. Paul, Minn.) and the tumors grown for 8 days. For all intravenous injections, mice were gently warmed with a heat lamp, placed on a restrainer, tail sterilized with alcohol pads, and the injection was placed into the lateral tail vein. All animal experiments were done in accordance with protocols approved by the Institutional Animal Care and Use Committee of MSKCC and followed National Institutes of Health guidelines for animal welfare.

### Serum Stability

A sample of the corresponding radiolabeled liposomal preparation (typically 1.9-2.6 MBq in 40-60 μL) was added to 400 μL of FBS. The mixture was incubated at 37° C. for 5 days. Aliquots of 0.3-0.4 MBq were taken at predetermined time points for size exclusion chromatography analysis by careful integration of the peaks.

### Blood Half-Life

Healthy female NCr mice (8-10 weeks old and 15-20 g of weight, n=6) were injected with 0.2-0.3 MBq (3-4 μmol lipid) of liposome preparation in 200 μL PBS solution. Blood was sampled from saphenous vein at predetermined time points (5 min, 30 min, 2 h, 6 h, 20 h and 26 h) and radioactivity measured on a Wizard2 2470 Automatic Gamma Counter (Perkin Elmer). Measurements were carried out in triplicate and the radioactivity content was calculated as the mean percentage injected dose per gram tissue (% ID/g)±S.D.

### Biodistribution Studies

Biodistribution experiments were conducted on female NCr nude mice (6-10 weeks old and 15-20 g of weight, n=21) bearing 4T1 breast xenografts. The radiolabeled liposome preparation (0.6-0.8 MBq, 0.4-0.6 μmol lipid, in 200 μL PBS solution) was administered via the lateral tail vein, and allowed to circulate for various time points (2 h, 24 h and 48 h), after which the mice were sacrificed and the organs perfused. The radioactive content in tissues of interest, (blood, tumor, large and small intestines, stomach, kidneys, brain, bone, liver, lungs, heart, skin, spleen, bladder, tail) was measured using a 2470 Wizard Automatic Gamma Counter (Perkin Elmer) and the tissue associated activity was calculated as the mean percentage injected dose per gram of tissue (% ID/g).

### Autoradiography

Following sacrifice, liver, spleen, tumor and muscle tissues were excised and embedded in OCT mounting medium (Sakura Finetek, Torrance, Calif.), frozen on dry ice, and a series of 10 μm frozen sections cut. To determine radiotracer distribution, digital autoradiography was performed by placing tissue sections in a film cassette against a phosphor imaging plate (BASMS-2325, Fujifilm, Valhalla, N.Y.) for 48 h at −20° C. Phosphor imaging plates were read at a pixel resolution of 25 μm with a Typhoon 70001P plate reader (GE Healthcare, Pittsburgh, Pa.). After autoradiographic exposure, the same frozen sections were then used for immunohistochemical staining and imaging.

### Staining/Microscopy

Tissue sections (10 μm, frozen) were stained for Iba1 with anti-Iba1 rabbit polyclonal antibody (Wako, Richmond, Va.) followed by a biotinylated goat anti-rabbit secondary antibody (VECTASTAIN® ABC kit, Vector Labs, Burlingame, Calif.), followed by Alexafluor 568-tyramide (Carlsbad, Calif.) for fluorescent signal (VECTASTAIN® ABC kit, Vector Labs, Burlingame, Calif.). Additional DAPI staining was performed using 4′,6-Diamidino-2-phenylindole dihydrochloride (Sigma Aldrich, St. Louis, Mo.). All sections were counterstained with hematoxylin & eosin (H&E) solution. All images were obtained using an EVOS FL Auto digital inverted fluorescence microscope (Life Technologies). Fluorescent images were obtained at the 4× objective while brightfield images were obtained at both the 4× and 20× objectives. On stained sections, Iba1 fluoresence was observed using a Texas Red light cube (Ex 585/29, Em 624/40, EVOS LED Light cube, Life Technologies), while DAPI fluorescence was observed using a DAPI light cube (Ex 357/44, Em 447/60, EVOS LED Light cube, Life Technologies). On sections containing DilC, fluorescence was observed using a Cy5 light cube (Ex 628/40, Em 692/40, EVOS LED Light cube, Life Technologies).

### PET/CT Imaging

Female Nude NCr mice (8-10 weeks old, n=8) bearing 4T1 breast tumors were injected with 7.5-9.3 MBq [⁸⁹Zr]liposomes (3-4 μmol lipid) in 200-250 μL PBS solution via the lateral tail vein. At predetermined time points (2 h, 24 h, 48 h and 120 h) animals were anesthetized with isofluorane (Baxter Healthcare, Deerfield, Ill.)/oxygen gas mixture (2% for induction, 1% for maintenance) and scans were then performed using an lnveon PET/CT scanner (Siemens Healthcare Global). Whole body PET static scans recording a minimum of 50 million coincident events were performed, with duration of 10-20 min. The energy and coincidence timing windows were 350-700 keV and 6 ns, respectively. The image data were normalized to correct for nonuniformity of response of the PET, dead-time count losses, positron branching ratio, and physical decay to the time of injection, but no attenuation, scatter, or partial-volume averaging correction was applied. The counting rates in the reconstructed images were converted to activity concentrations (percentage injected dose [% ID] per gram of tissue) by use of a system calibration factor derived from the imaging of a mouse-sized water-equivalent phantom containing ⁸⁹Zr. Images were analyzed using ASIPro VM™ software (Concorde Micro-systems). Quantification of activity concentration was done by averaging the maximum values in at least 5 ROIs drawn on adjacent slices of the tissue of interest. Whole body standard low magnification CT scans were performed with the X-ray tube setup at a voltage of 80 kV and current of 500 μA. The CT scan was acquired using 120 rotational steps for a total of 220 degrees yielding and estimated scan time of 120 s with an exposure of 145 ms per frame.

### Near Infrared Imaging

Fluorescence imaging was performed on an IVIS Spectrum (Caliper) system (Perkin Elmer). Fluorescence images were acquired with excitation and emission wavelengths 650 and 670 nm, respectively, and using auto acquisition times. Data were quantified as radiant efficiency.

### Preparation of Liposomes

All liposome preparations used in the present work were obtained by the sonication method. Briefly, a lipid film was prepared by evaporating a chloroform solution containing the corresponding lipids in the desired proportion. The resulting film was hydrated with PBS (typically 10 ml) and sonicated for 25 min using a Biologics, Inc 150 V/T Ultrasonic Homogenizer working at 30% power output. After quick centrifugation, size and Z-potential measurements were performed on a Malvern NanoSeries Z-Sizer and a Zeta PALS analyser (Brookhaven Instruments Corporation) respectively. Liposomes containing the synthesized lipids were concentrated using a Millipore 100 kDa VivaSpin tube and washed twice with PBS. Fluorescent liposomes were prepared in the same fashion, including the dye in the initial chloroform solution.

### Synthesis of 3-{({(23-(11,12-dehydrodibenzo[b,f]azocin-S(GH)-yl)-4,20,23-trioxo-7,10,13,16-tetraoxa-3,19-diazatricosyl)oxy)(hydroxy)phosphoryl)oxy)propane-1,2-diyldistearate

A solution of DBCO NHS ester (2, 12.8 mg, 19.7 μmol), 1,2-distearoyl-sn-glycero-3-phosphoethanolamine (DSPE, 1, 16.2 mg, 21.7 μmol), and diisopropylethyl amine (5.4 μL) in dry dichloromethane (2 ml) was prepared in a round bottom flask equipped with a condenser. The system was purged with nitrogen and the mixture stirred at 40° C. for 15 hours. The resulting solution was chromatographed on silica gel, using gradient elution from neat dichloromethane to dichloromethane/methanol 5:1 to obtain the desired product as a pale yellow solid (14.4 mg, 57% yield). 1H-NMR (CDCb): 0.90 (t, 6H), 1.26 (bs, 56H), 1.60 (bs, 4H), 2.31 (t, 4H), 2.54 (m, 4H), 3.32 (m, 2H), 3.47 (m, 4H), 3.58 (m, 12H), 3.57 (d, 1H), 3.71 (t, 2H), 3.99 (bs, 4H), 4.10 (m, 1H), 4.36 (m, 1H), 5.07 (d, 1H), 5.15 (m, 1H), 7.23 (m, 2H), 7.29 (m, 2H), 7.36 (m, 3H), 7.58 (m, 1H), 7.64 (m, 1H), 11.3 (bs, 1H). HRMS TOF ES [Mr: m/z 1280.8057 (calculated for C71H115N301sP 1280.8066).

### Synthesis of N′-(1-azido-15-oxo-3,6,9,12-tetraoxa-16-azahenicosan-21-yi)-N1-hydroxy-N″-(5-(N-hydroxy-4-((5-(N-hydroxyacetamido)pentyl)amino)-4-oxobutanamido)pentyl)-succinamide

A suspension DFO mesylate (5, 33.8 mg, 5 μmol), PEG4-azide NHS ester (4, 20 mg, 5 μmol) and diisopropylethyl amine (15.0 μL) in dry dimethylformamide (DMF, 0.7 ml) was stirred at 40-45° C. for 7 hours under nitrogen. After cooling down to room temperature, diethyl ether (1 ml) was added and the mixture was kept at 4° C. for another hour. The solid was then filtered and washed thoroughly with methanol to furnish the pure product as a white solid (6.5 mg, 15% yield). 1H-NMR (CD300): 1.31 (m, 6H), 1.48 (m, 6H), 1.59 (m, 6H), 2.05 (s, 3H), 2.40 (m, 6H), 2.72 (m, 4H), 3.12 (m, 6H), 3.39 (t, 2H), 3.56 (m, 6H), 3.61 (m, 12H), 3.63 (m, 2H), 3.68 (t, 2H). HRMS TOF ES [M+Naf: m/z 856.4724 (calculated for C36H67N9013Na 856.4756).

### Synthesis of 3-((hydroxy(2-(3-(4-(3-(3,14,25-trihydroxy-2,10,13,21,24-pentaoxo-3,9,14,20,25-pentaazatri-acontan-30-yl) thioureido)phenyl)thioureido)ethoxy)phos-phoryl)oxy)propane-1,2-diyldi-stearate

To a solution of DFO-NCS (7, 6.0 mg, 8.0 μmol) in dimethyl sulfoxide (0.5 ml) was added 1,2-distearoyl-sn-glycero-3-phosphoethanolamine (DSPE, 1, 9.9 mg, 13.2 μmol), chloroform (0.5 ml) and diisopropylethyl amine (20.0 μL). The mixture was stirred at 50° C. under nitrogen for three days, after which time chloroform was evaporated and 1 M Tris was added (0.5 ml). Half an hour later, the suspension was filtered and the resulting solid washed with 1 M Tris (2×1 ml), water (3×1 ml)and dichloromethane (3×1 ml). The white solid was dried to afford the title product (11.1 mg, 85%). 1H-NMR (Tris salt, CDCl3/DMSO-d6):0.91 (t, 6H), 1.25 (bs, 62H), 1.37 (m, 4H), 1.52 (m, 12H), 1.99 (s, 3H), 2.22 (m, 4H), 2.35 (m, 4H), 2.64 (m, 4H), 3.04 (m, 4H), 3.47 (bs, 8H), 3.51 (s, 6H), 3.68 (m, 2H), 3.82 (bs, 2H), 3.88 (bs, 2H), 4.05 (m, 1H), 4.28 (m, 1H), 5.07 (m, 1H), 5.10 (bs, 3H), 7.22 (d, 2H), 7.34 (bs, 3H), 7.59 (s, 1H), 7.66 (bs, 4H), 7.72 (bs, 1H), 9.23 (s, 1H), 9.50 (bs, 1H), 9.69 (s, 1H), 9.76 (s, 1H), 9.82 (bs, 1H). HRMS FAB [M−Hr: m/z 1498.8977 (calculated for 4H133N9016PS2 1498.9049).

### Synthesis of [⁸⁹Zr]9

Precursor 6 (5-10 μg in DMSO) was dissolved in PBS (200 μL). Activity ([89Zr]zirconium oxalate in 1 M oxalic acid) was added followed by addition of an equal volume of 1 M sodium carbonate to adjust pH to 7.1-7.4. The solution was vortex-mixed and reacted at 40° C. for 30 minutes. The product was purified by HPLC using a C4 Phenomenex Jupiter column, using solvents A and C, and gradient elution from 5 to 100% C over 27 minutes. The retention time of [⁸⁹Zr]9 was 16.1 min and its identity was established by coelution with the reference cold compound (FIG. 11B). The radiochemical yield was 44±15% (n=7) and the radiochemical purity greater than 98%. The collected fraction containing [⁸⁹Zr]9 was concentrated to dryness in vacuo and used in the following step.

### Synthesis of 89Zr-CLL

Over the isolated [⁸⁹Zr]9 (18-37 MBq) a solution of DBCO-L (typically 0.5-1.0 ml, 5-10 μmol lipid) in PBS was added. A 1 M sodium carbonate solution was used to adjust pH to 6.8-7.4. The mixture was sonicated and shaken at 400 rpm at 30° C. in a Thermomixer for 16 h and finally loaded onto a 100 kDa NMWL Amicon centrifugal filter (Millipore). The solution was concentrated by centrifugation at 4000 g and washed at least twice with 0.5 mlPBS, until the activity in the filtrate was constant. The resulting concentrate was diluted with PBS to the desired volume. The radiochemical yield was 14±4% (n=3) and the radiochemical purity greater than 95%, as established by size exclusion chromatography. DLS measurements were also performed.

### Synthesis of 89Zr-SCL

To a solution of DFO-L (typically 0.5-1.0 ml, 5-10 μmol lipid) in PBS was added [⁸⁹Zr]zirconium oxalate in 1M oxalic acid (18-37 MBq), followed by addition of an equal volume of 1 M sodium carbonate to adjust pH to 7.1-7.4. The mixture was shaken at 400 rpm at 30° C. in a Thermomixer for 4 h and finally loaded onto a 100 kDa NMWL Amicon centrifugal filter (Millipore). The solution was concentrated by centrifugation at 4000 g and washed at least twice with 0.5 mlPBS, until the activity in the filtrate was constant. The resulting concentrate was diluted with PBS to the desired volume. The radiochemical yield was 74±10% (n=7) and the radiochemical purity greater than 99%, as established by size exclusion chromatography. DLS measurements were also performed.

### Discussion of Example 3

The size of all liposomes was larger than the renal clearance threshold. The overall negative charge of the particles helps to stabilize them in vivo, reducing the tendency to aggregate and avoiding electrostatic interactions with the luminal wall of blood vessels. Moreover, the presence of polyethylene glycol chains on the surface of the particles efficiently helps to prevent opsonization and their subsequent removal from circulation.

Compared to the bioorthogonal labeling, direct surface chelation proved to be quicker and more efficient, but neither strategy had a significant effect on the size distribution of the samples when compared to their precursor liposomes. Although both probes had a similar size, the clearance rates of radioactivity from blood were significantly different. After 24 h, 7.1% ID/g was remaining in the blood pool for ⁸⁹Zr-SCL whereas the activity for ⁸⁹Zr-CLL had dropped to 1.5% ID/g.

Circulation time is a critical factor for tumor accumulation, especially for probes reliant on passive targeting mechanisms. Higher accumulation is to be expected for those species showing the longest half-lives, as more particles will extravasate into the interstitial space with higher passage numbers. The fast blood clearance observed for ⁸⁹Zr-CLL can be explained by the rapid accumulation of radioactivity in liver and spleen, which are the organs that most efficiently remove particles from circulation. In contrast, ⁸⁹Zr-SCL seemed to evade the mononuclear phagocyte system (MPS) longer and therefore had substantially lower liver and spleen uptake at 2 hours post injection. This difference could be due to a higher tendency of ⁸⁹Zr-CLL to aggregate into larger particles and their subsequent removal by the MPS, and illustrates how changes in the surface chemistry of nanoparticles can have far-reaching consequences to their stability and pharmacokinetic profile. As a result of this, tumor accumulation for ⁸⁹Zr-SCL was dramatically higher than for their click-labeled counterparts ⁸⁹Zr-CLL at all time points, peaking at 24 h post-injection (13.7±1.8% ID/g and 2.0±0.2% ID/g, respectively). Tumor-to-blood uptake ratios for ⁸⁹Zr-SCL were 0.09, 2.0 and 4.2 at 2 h, 24 h and 48 h, respectively. Interestingly, no correlation was found between size of tumor and uptake (% ID/g) for either probe. For both liposomes, bone uptake was moderate (less than 6% ID/g at 48 h), which indicates that the ⁸⁹Zr-OFO metal complex in the liposome surface is protected from trans-chelation and release of the radiotracer. These data also indirectly support the very low unspecific adsorption of ⁸⁹Zr on the surface of ⁸⁹Zr-SCL, as liposomes labeled in the absence of OFO or other chelators have poorer in vitro and in vivo stabilities and, consequently, higher bone uptake. The data presented here compare very well with reported bone uptake values for other long-circulating ⁸⁹Zr-labeled probes.

PET/CT imaging mirrored the results of the biodistribution studies for both liposomes as shown in mice bearing either xenografted or orthotopically implanted 4T1 breast tumors. While very low tumor uptake could be observed for ⁸⁹Zr-CLL, an intense and persistent signal was found in all tumors imaged with ⁸⁹Zr-SCL at 24 h (FIG. 13C) and subsequent time points. There was no statistically significant difference observed between uptake in xenografts and orthotopically implanted tumors at 24 hours after administration of 89Zr-SCL (13.1±1.8% ID/g, n=4 and 12.2±3.4% ID/g, n=3, respectively). PET-quantified bone uptake was also similar to the biodistribution data for both formulations, and was found to be lower than 5% ID/g at all time points, even after 120 h. The radioisotope ⁸⁹Zr is a bone seeker that eventually will accumulate in the mineral bone if released from its chelator OFO. A second pathway that might result in bone accumulation of nanoparticle-bound radiotracers is their uptake in macrophages. The presence of tissue macrophages in the bone marrow as part of the MPS makes it a potential undesired destination for radiolabeled nanoparticles.

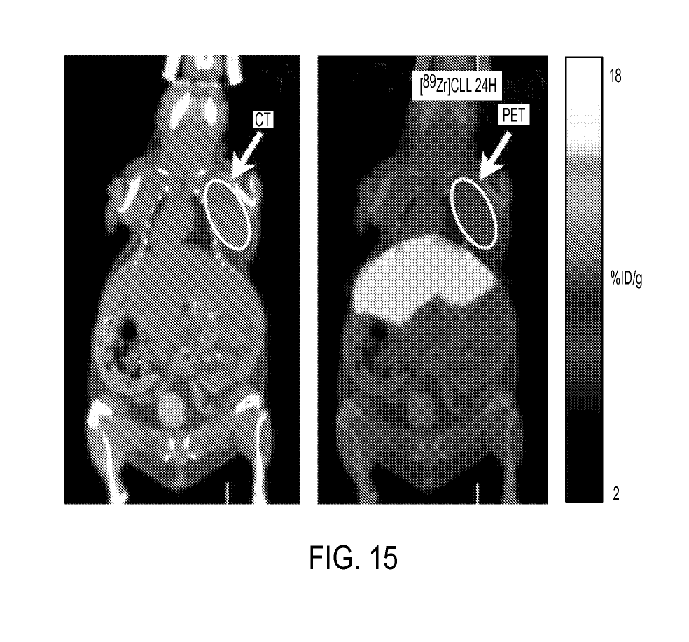

The distribution of ⁸⁹Zr-SCL in the tumors was not homogeneous (FIG. 13C). In tumors grown for over 10 days, two regions were clearly distinguishable: a peripheral shell with high accumulation (as high as 20% ID/g); and a central core, showing low tracer uptake. Histological analysis confirmed these findings (FIG. 15), and there is evidence for localization of ⁸⁹Zr-SCL to macrophage-rich areas, as reported for other nanoparticulate systems in varied disease models.

Encouraged by the results obtained with ⁸⁹Zr-SCL, a bimodal PET/optical imaging agent was generating by adding a NIR fluorescent dye (DilC) to the lipid formulation. Although tumor uptake for DilC@⁸⁹Zr-SCL as measured by PET was lower (10.8±2.1% ID/g, n=3) than that of ⁸⁹Zr-SCL (14.1±1.6% ID/g, n=4), this difference was not statistically significant (p=0.10). The high level of co-localization of both signals (FIG. 16C), as well as the quantitative analyses by PET and NIR fluorescence imaging (FIG. 16D), suggest a good stability of these liposomal nanoparticles.

What is claimed is:

1. A desferrioxamine-bearing liposome (DFO-L) labeled with ⁸⁹Zr and a fluorophore, wherein the ⁸⁹Zr is attached to a surface of the liposome via a chelating moiety.

2. The liposome of claim 1,
wherein the chelating moiety is lipid-based and/or comprises a lipophilic anchor group, and
wherein the chelating moiety is a phospholipid-chelator.

3. The liposome of claim 2, wherein the chelating moiety comprises 1,2-distearoyl-sn-glycero-3-phosphoethanolamine (DSPE)-desferrioxamine (DFO).

4. The liposome of claim 1, wherein the fluorophore comprises a NIR (near infrared) dye.

5. The liposome of claim 4, wherein the NIR dye is Cy5 or Cy7.

6. A dibenzoazacyclooctyne-bearing liposome (DBCO-L) labeled with ⁸⁹Zr and a fluorophore, wherein the ⁸⁹Zr is attached to a surface of the liposome via a clickable moiety.

7. The liposome of claim 6,
wherein the clickable moiety comprises one or more of a DFO-azide group or a bioorthogonal group, and
wherein the bioorthogonal group comprises one or more of trasn-cyclooctene, tetrazine, alkyne, strained alkene, thiol, DFO-azide, and maleimide.

8. The liposome of claim 6, wherein the fluorophore comprises a NIR dye, the NIR dye being Cy5 and/or Cy7.

9. The liposome of claim 1, wherein the liposome has a mean diameter from about 10 nm to about 1 μm

10. The liposome of claim 1, wherein liposome has a mean diameter of from 25 nm to 500 nm, from 50 nm to about 300 nm, from 75 nm to 150 nm, from 10 nm to 25 nm, or from 500 nm to 1 μm.

11. A method of treating a disease or disorder, the method comprising:
administering a desferrioxamine-bearing liposome (DFO-L) labeled with ⁸⁹Zr and a fluorophore to a subject, wherein the ⁸⁹Zr is attached to a surface of the liposome via a chelating moiety.

12. The method of claim 11, wherein the chelating moiety is lipid-based and/or comprises a lipophilic anchor group, the chelating moiety being a phospholipid-chelator or 1,2-distearoyl-sn-glycero-3-phosphoethanolamine (DSPE)-desferrioxamine (DFO).

13. The method of claim 11, wherein the fluorophore comprises a NIR (near infrared) dye, the NIR dye being Cy5 and/or Cy7.

14. The method of claim 11, further comprising:
capturing and displaying one or more of:
(i) a positron emission tomography (PET) image of a tissue of the subject comprising the radiolabeled liposome;
(ii) an optical image of a tissue of the subject comprising the radiolabeled liposome;
(iii) a sequence of PET images in real time; and
(iv) a sequence of optical images in real time, the sequence of optical images being a sequence of fluorescence images.

15. The method of claim 14, wherein the capturing and displaying the positron emission tomography (PET) image of a tissue of the subject comprising the radiolabeled liposome and the capturing and displaying the optical image of the tissue of the subject comprising the radiolabeled liposome are performed contemporaneously.

16. The method of claim 14, wherein the capturing and displaying the positron emission tomography (PET) image of a tissue of the subject comprising the radiolabeled liposome and the capturing and displaying the optical image of the tissue of the subject comprising the radiolabeled liposome are conducted during a surgical procedure.

17. A method of testing loading and/or delivery potential of a bimodal-labeled liposome in a tissue of a subject, the method comprising:
(a) administering the bimodal-labeled liposome, wherein the bimodal-labeled liposome is labeled with a radioisotope and a near infrared (NIR) dye, wherein the NIR dye comprises a lipophilic drug-mimic to test loading and/or delivery potential of the liposome;
(b) capturing and displaying a positron emission tomography (PET) image of the tissue of the subject comprising the radiolabeled liposome; and
(c) capturing and displaying an optical image of the tissue of the subject comprising the radiolabeled liposome.

18. The method of claim 17,
wherein the bimodal-labeled liposome is a desferrioxamine-bearing liposome (DFO-L) labeled with ⁸⁹Zr and a fluorophore,
wherein the ⁸⁹Zr is attached to a surface of the liposome via a chelating moiety,
wherein the chelating moiety is lipid-based and/or comprises a lipophilic anchor group, and
wherein the chelating moiety is a phospholipid-chelator.

19. The method of claim 18, wherein the chelating moiety is 1,2-distearoyl-sn-glycero-3-phosphoethanolamine (DSPE)-desferrioxamine (DFO).

20. The method of claim 17, further comprising capturing and displaying a sequence of PET images in real time.

21. The method of claim 17,
wherein the NIR dye comprises Cy5 and/or Cy7, and
wherein the optical image comprises a fluorescence image.

22. The method of claim 17, further comprising capturing and displaying a sequence of optical images in real time.

23. The method of claim 17, wherein capturing and displaying a first PET image is performed at 24 hours after administration.

24. The method of claim 17, further comprising
(d) administering a second bimodal-labeled liposome comprising a therapeutic, wherein the bimodal-labeled liposome is labeled with a radioisotope and a fluorophore.

25. The method of claim 24, wherein the bimodal-labeled liposome is a desferrioxamine-bearing liposome (DFO-L) labeled with ⁸⁹Zr and a fluorophore, wherein the ⁸⁹Zr is attached to a surface of the liposome via a chelating moiety.

26. The method of claim 25, wherein the chelating moiety is one or more of:
(i) lipid-based comprising a lipophilic anchor group;
(ii) a phospholipid-chelator; and
(iii) 1,2-distearoyl-sn-glycero-3-phosphoethanolamine (DSPE)-desferrioxamine (DFO).

27. The method of claim 24, wherein the therapeutic comprises a cytotoxic drug, doxorubicin.

28. The method of claim 25, wherein the second biomodal-labeled liposome comprises taxol or doxorubicin HCl liposome.

29. The method of claim 25,
wherein the fluorophore comprises a NIR dye, and
wherein the NIR dye is Cy5 and/or Cy7.

30. The method of claim 25, further comprising:
(e) capturing and displaying a positron emission tomography (PET) image of the tissue of the subject comprising the radiolabeled liposome comprising the therapeutic; and/or
(f) capturing and displaying an optical image of the tissue of the subject comprising the radiolabeled liposome comprising the therapeutic.
